(19) United States
(12) Patent Application Publication
(10) Pub. No.: US 2021/0038730 A1
(43) Pub. Date: Feb. 11, 2021

(54) PEPTIDE NANOFIBERS

(71) Applicant: UNIVERSITY OF PORTSMOUTH HIGHER EDUCATION CORPORATION, Portsmouth Hampshire (GB)

(72) Inventors: Aikaterini LALATSA, Portsmouth Hampshire (GB); Diane LEITE, Portsmouth Hampshire (US)

(21) Appl. No.: 17/045,085

(22) PCT Filed: Apr. 2, 2019
(86) PCT No.: PCT/GB2019/050950

§ 371 Date: Oct. 2, 2020

(30) Foreign Application Priority Data
Apr. 3, 2018 (GB) 1805455.1

(51) Int. Cl. A61K 47/42; A61K 47/64; C07K 7/06; A61K 31/337; A61K 31/495; A61K 31/704

(52) U.S. Cl. CPC A61K 47/42; A61K 47/64; C07K 7/06; A61K 45/06; A61K 31/495; A61K 31/704; A61K 31/337

(57) ABSTRACT

a nanofiber comprising a peptide GPCR modulator conjugated to a lipophilic moiety where in the peptide-lipophilic moiety conjugate comprises a poly(proline) type II helix structure.

The invention relates to a system for the delivery of hydrophilic peptides and other drugs to the brain and tumours.

The therapy of brain diseases, such as pain, migraine, neurodegenerative conditions, mood disorders and stroke, cancers and metastatic cancers still remains challenging. Peptides are of tremendous clinical value for the treatment of many tumours or central nervous system (CNS) disorders, especially due to their high potency, high specificity and low inherent toxicity. Many existing peptide pharmaceuticals are rendered ineffective after parenteral, nasal, transdermal or oral administration due to enzymatic instability and inability to cross biological barriers, such as the blood-brain barrier (BBB), nasal epithelium, gastrointestinal tract epithelium and stratum corneum due to their hydrophilicity, size and charge.

Neuropeptide receptors belong almost exclusively to the G-protein coupled receptor (GPCR) family of receptors. The GPCR super family is the largest and single most important family of drug targets in the body. Neuropeptides act on specific cell membrane receptors of the GPCR super family and are essential modulators of a range of important aspects of signal transduction, neurotransmission, ion channel regulation and other cellular nervous and endocrine functions, and are linked to a wide range of disease areas. GPCRs are expressed in every type of cell in the body where their function is to transmit signals from outside the cell across the membrane to signalling pathways within the cell, between cells and between organ systems. GPCRs are divided into five classes and the majority of them are comprised of a short extracellular N-terminal domain, seven transmembrane α-helices, connected by three intra- and extra cellular loops and an intracellular C-terminus. They are activated by a diverse range of ligands and stimuli, including hormones, neurotransmitters, membrane lipids, ions and light.

GPCRs are important molecular targets for pharmacological intervention for a quarter of marketed drugs. High-value targets are, for example, non-aminergic GPCRs that are activated by peptides (in comparison to aminergic GPCRs that are activated by neurotransmitters such as serotonin). Extracellular binding sites for non-aminergic GPCRs negate larger molecular weight ligands in order to interact with the lower relatively accessible surface area. Thus, many of the hydrophilic, enzymatically unstable high molecular weight (>500 Da) peptide ligands, possess unfavourable physicochemical characteristics requiring parenteral administration and are unable to cross biological barriers and membranes including the blood-brain barrier, to modulate their respective GPCRs.

There is therefore a need to identify new systems for the delivery of such hydrophilic drugs to, for example, the brain and to tumours.

WO 2012/004610 describes producing conjugates of peptides attached to hydrophobic moieties such as fatty acids. These have been observed to form nanofibers. A single peptide, delargin, has been exemplified as forming nanofiber structures and was capable of being detected in the brain of animals after intravenous administration.

U.S. Pat. No. 7,695,7228 relates to the detection of gonadotrophin-hormone releasing hormone (GnRH) receptors in the brain or nervous system. GnRH agonists are also described. Where tumours are within the blood-brain barrier, direct injection is proposed or intra-arterially into the nervous system. Conjugates of GnRH peptide agonists having at least 10 amino acids on length are discussed, in combination with gonadotrophin or luteinising hormone inhibitors. Other peptides having at least 11 amino acids on length for treating such diseases are also discussed in WO 2005/116058.

Neuropeptide GPCRs modulators such as GnRH are found in cancers, such as glioblastoma multiforme, breast cancer, melanoma, pancreatic cancers, lung cancer, colorectal, ovarian, bladder, endometrial and prostate cancers and their metastatic cancers including central nervous system metastatic tumours. Accordingly, such modulators are of particular interest.

The inventors have now recognised that the ability to deliver such peptide GPCR modulators to tumours and across the blood-brain barrier to, for example the brain, would be useful. Additionally, if it is possible to deliver such peptides in combination with one or more additional non-peptide drugs, then this would increase the ability of the physician to specifically target the disease of interest and to treat the disease with two drugs at the same time.

The invention therefore provides a nanofiber comprising a peptide GPCR modulator, conjugated to a lipophilic moiety, optionally via a selectively cleavable link, the nanofiber comprising a poly(proline) type II helix structure, optionally additionally comprising one or more additional bioactive compounds, such as drugs or biomacromolecules, or imaging moieties. The GPCR modulator may be conjugated to the lipophilic moiety via a covalent bond, which may be selectively cleavable.

The bioactive compound such as drug or biomacromolecule or imaging moiety is typically entrapped, conjugated, or complexed within the nanofiber or adsorbed onto the surface of the nanofiber.

Nanofibers are long-axial fibres with at least one dimension in the nanometre range, such as 1-1000 nm (typically 5-100 nm) and lengths of up to 20 microns (typically 0.2-2 μm) as supported by for example TEM and AFM studies. The amphiphilic nature of the peptide allows nanostructures to be formed. When such nanofibers are formed, they have been found to remain stable at different temperatures, for example 15-40° C.

The inventors have now found that upon further dilution in aqueous media, the fibres form polyproline type II helix structures, in contrast to previous reports. Previously, peptide nanofibers were reported to be formed from β-sheet forming peptide chains which could be linked to a hydrophobic group forming a central hydrophobic core.

Polyproline II (PP II) helices are a type of protein secondary structure which occurs in proteins which often contain proline residues. A left handed polyproline II helix is formed when sequential residues adopt a backbone with average dihedral angles of typically −75° and 145° or 150°, and typically have trans-isomers of their peptide bonds. Each PP II helix usually has 3 residues per turn compared to 3-6 residues in an α-helix.

Pro, but also Gln, Asp, Gly, Ala and Leu have been shown to have high PP II properties (Rath et al, Biopolymers (2005) 80, 179-185).

Typically the peptide GPCR modulator comprises at least one Pro, Gln, Asp, Gly, Ala or Leu residue, and most typically contains at least one Pro residue

Typically the length of the peptide is less than 40 or less than 30 amino acids, most typically less than 15 or less than 11 amino acids long, and may contain at least 5 or at least 6 amino acids in length.

The GPCR modulator is typically selected from a gonadotrophin hormone releasing hormone (GnRH) receptor binding peptide, angiotensin 1-7, an opioid neuropeptide, neuropeptide S, neuropeptide Y, a gastrin releasing peptide, orexin, dynorphin, detorphin I, oxytosin, vasopressin, leptin, enkephalin, met-enkephalin, tyr-enkephalin, urotensin II-Related Peptide (URP), urotensin II, vasoactive intestinal peptide, substance P, somatostatin peptide, intermedin, urocortin 1, brain natriuretic peptide, and secretin. More typically the peptide is a GnRH receptor binding peptide.

This may be selected from pyroGlu-His-Trp-Ser⁴-Tyr⁵-Gly⁶-Leu-Arg-Pro-Gly-NH₂ (GnRH), Glu-His-Trp-Ser⁴-Tyr⁵-Gly⁶-Leu-Arg-Pro-Gly-NH2 (Glu-GnRH) and Tyr-Gly-Leu-Arg-Pro-Gly-NH₂ (Tyr-GnRH), or H-Ala-Gly-Cys-Lys-Asn-Phe-Phe-Trp-Lys-Thr-Phe-Thr-Ser-Cys-OH (Cys3-Cys14).

Typically the drug is a non-peptide drug. Examples include paclitaxel, docetaxel, temozolomide, doxorubicin, lomustine, etoposide, carmustine, cisplatin, buparvaquone, atovaquone, lonidamine and a polynucleotide, or mixtures thereof. The drug may be an antigen or a toxoid to allow the nanofiber to act as a vaccine and may be a peptide, such as 4N1K peptide (KRFYVVMWKK), which is derived from the C-terminal cell-binding domain of thrombospondin-1 (TSP-1). The drug may be an LDH inhibitors and particularly LDH-A inhibitors e.g. NHI2, Oxamate, FX11, Galloflavin, Quinoline 3-Sulfonamides derivatives, Stiripentol (and its derivatives), Gossypol (and its derivatives), Mn(II) complexes. (See Valvona, C, Fillmore, H L, Pilkington, G J, Nunn, PB Brain Pathology 2015. The regulation and function of lactate dehydrogenase A: Therapeutic Potential in Brain Tumor (doi:10.1111/bpa.12299).

Phenformin, Metformin and radio sensitizers may also be provided. The radio sensitizer may be used to assist in the treatment of brain tumours by radiotherapy.

The polynucleotide may, for example, encode a peptide or protein product for gene therapy or encode an inhibitor of one or more genes. Examples of such nucleotides includes DNA, RNA, mRNA, siRNA, and shRNA.

The imaging moiety may be a visually infra-red or ultra-red violet detectable moiety such as a fluorescent molecule such as fluorescein or a pigment. It may be a spion, an MRI contrast agent (such as a gadolinium, iron, iron-platinum or manganese containing compound); or a RAMAN tag for CARS microscopy. The lipophilic moiety itself may be deuterated to allow the nanofiber to be detected by CARS.

The ability to combine the drug with the complex means that there is lower haemolytic toxicity with the drugs loaded onto the conjugate, compared to the free drug within the system. Additionally, there is excellent stability of the drug with the conjugate, and the drug with the conjugate has been shown by the inventors to have the ability to transfer across the blood-brain barrier when loaded with nanofibers, but not as free drug.

Moreover, the drug is targeted, as it binds to the GPCR receptor. This allows a lower level of the drug to be used, resulting in lower toxicity to the patients as a whole. Additionally, they may be able to have anti-proliferative activity using different pathways to cell apoptosis.

Drug loaded nanofibers can be formulated by a variety of methods generally known in the art including probe sonication and rotor-evaporation or electrostatic complexation.

The peptide GPCR modulator may be modified, for example at the C-terminal end to possess a free C-terminal, (R—COOH) or an amidated C-terminal (R—CONH2) or an alkylated C-terminal (R—COO—R1). Conjugating an aromatic saccharide amphiphile or one or more amino acids to the C-terminus can cause the nanofiber to gel. Gelation can also be induced by changes in pH, salts, osmolality. Alternatively, one or more gelling agents may be mixed with or conjugated to the nanofiber. Such gelling agents include hydrogels such as alginates, chitosans, fibrin actin, silk fibroin, hyaluronic acid or mixtures thereof. This allows so called bioinks to be produced. Bioinks are materials that mimic the extracellular matrix environment to support adhesion, proliferation and differentiations of living cells. They may be printed, for example, via 3D printing into 3D shapes prior to use or implantation. Printed articles comprising such bioinks are therefore also provided.

Fleming S. and Ulijn R. (Chem. Soc. Rev. 2014, 43, 8150-8177) and V. Ryan D. M. et al (Langmuir, 2011, 27-4029-4039), for example, describe modifying the C-terminus of peptides can affect gelation. Varying the C-terminus of various side chain halogenated Fmoc-phenylalanine derivatives has broadly revealed that COOH promotes gelation, COOMe promotes precipitation, and CONH2 generally results in solutions. Aromatic saccharide amphiphiles may also be added. Gelation can also be induced by changes in pH, salts, osmolality or adding short peptide sequences or serine to the C terminus (Ozbas B. et al., Macromolecules, 2004, 37, 7331-7337 and Anderson J. M. et al. ACS Nano. 2009, 24, 3447-3454).

Compositions comprising a nanofiber according to the invention modified with, or in combination with, a gelling agent as defined above, are also provided.

The nanofibers may be embedded within, for example polysaccharide nanocrystals such as cellulose nanocrystals.

Bioinks, autogels and implants comprising the nanofibers are also provided.

Lipophilic typically means a compound having a very low solubility in water, such as below 0.1 mg/L. Hydrophilic typically means a compound that has a high water solubility (typically > than 1 mg/ml).

The nanofiber may be conjugated with, for example, a detectable/imaging compound such as a fluorescent compound to allow the detection or passage of the nanofiber to be followed.

The group may comprise for instance a C6-30 alkyl group, a C6-30 acyl group, a multicyclic hydrophobic group with more than one C4-C8 ring structure such as a sterol (e.g. cholesterol, deoxycholic acid, ursodeoxycholic acid, ursolic acid), a multicyclic hydrophobic group with more than one C4-C8 heteroatom ring structure, a polyaxa C1-C4 alkylene group such as polyoxabutykene polymer, hydrophobic polymeric constituent such as poly(lactic acid) group, a poly(lactide-co-glucolide) group or poly(glycolic acid) group, or a lipidised D or L amino acid modified at their N terminal or side chain.

The hydrophilic group is more typically derived from a fatty acid, (so it contains a fatty acid moiety) derived from, for example, palmitic acid, (so it contains for example a palmitoyl group), caprylic, capric, lauric, myristic, stearic, arachidic, cholic, deoxycholic, ursodeoxycholic or ursolic acids. These may be deuterated.

The fatty acid may be attached, for example, to a D- or L-amino acid, to produce a lipidised D- or L-amino acid. This may be achieved, for example, by reacting a fatty acid ester attached to a reactive group, such as N-hydroxysuccinamide, which reacts with amine groups on the amino acids.

This latter example also allows the incorporation of an ester link. This may be used as the selectively cleavable linker, and may be broken by esterases soluble or membrane bound for example within a cell or plasma, to release the peptide.

Accordingly, the optional selectively cleavable link may for example be an ester or an amide link, with the nitrogen, oxygen or sulphur atom derived from the peptide. Alternatively, the linker may be releasable at lower pH, for example at pH of 1-5, which are sometimes found within local environmental conditions of the body such as within some tumours. Such selectively cleavable links are generally known in the art.

Typically the peptide is conjugated to a lipophilic group via a covalent bond.

Typically the peptide is conjugated to a lipophilic group at a part of the peptide backbone, or an amino acid side chain that it is not involved in the GPCRs binding.

The nanofibers may be overcoated or conjugated to long circulating polymers.

Such polymers are typically amphiphile coatings. They include, for example, chitosan. These have been observed to increase the plasma half-life of peptides in the circulation. Examples are disclosed in, for example, WO 2010/100470 and WO 2015/063510. Examples includes sorbitan esters, polysorbates, poly(ethylene glycol), typically at 2-5 kDa, carbohydrates such as chitosan polymers (typically 1-80 kDa) and their quaternary amine salts, glycol chitosan polymers (typically 1-80 kDa) and their quaternary amine salts, hyaluronic acid polymers (0.2-80 kDa) and hyaluronic acid-chitosan or glycol chitosan copolymers (typically 1-80 kDa), pullulan (typically 2.3-50 kDa), dextrans (typically 1-80 kDa), pectin (typically 1-80 kDa), guar gums (typically 1-80 kDa), alkyl glyceryl dextrans (typically 1-80 kDa), alginates (typically 1-80 kDa) cellulose and modified cellulose polymers and mixtures thereof.

The invention also provides pharmaceutical compositions comprising a nanofiber according to the invention. The composition may comprise a pharmaceutically acceptable carrier or excipient.

The compositions may be delivered to the human or animal body by a range of delivery routes including, but not limited to: gastrointestinal delivery, including orally and per rectum; parenteral delivery, including injection, patches, creams etc.; mucosal delivery, including nasal, inhalation and via pessary. In a preferred embodiment, the compositions are administered via parenteral (intravenous, subcutaneous, intramuscular), oral, nasal or topical routes (e.g. across the stratum corneum or bladder instillation) and most preferably nasally or by a parenteral route. Nasal, intravenous, intramuscular and subcutaneous routes may be especially used.

In addition to the peptide conjugate and amphiphile as described above, the pharmaceutical compositions may comprise a pharmaceutically acceptable excipient, carrier, diluent, buffer, stabiliser or other materials well known to those skilled in the art. Such materials should be non-toxic and should not interfere with the efficacy of the composition. The precise nature of the carrier or other material may depend on the route of administration, e.g. parenteral, intramuscular, subcutaneous, inserted into the bladder, oral, nasal or topical routes.

Pharmaceutical compositions for oral administration may be in tablet, capsule, powder or liquid form. A tablet may include a solid carrier such as gelatine or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. Surfactants, lipid esters and long-(LCT), medium-(MCT) and short-chain triglycerides (SCT) may also be included.

When tablets are used for oral administration, typically used carriers include sucrose, lactose, mannitol, maltitol, dextran, corn starch, typically lubricants such as magnesium stearate, preservatives such as paraben, sorbin, anti-oxidants such as ascorbic acid, alpha-tocopherol, cysteine, disintegrators or binders. When administered orally as capsules, effective diluents include lactose and dry corn starch. Liquids for oral use include syrups, suspensions, solutions and emulsions, which may contain a typical inert diluent used in this field, such as water. In addition, the composition may contain sweetening and/or flavouring agents.

For intravenous, intramuscular, cutaneous or subcutaneous injection, or injection at the site of affliction, the composition will be in the form of parenterally acceptable aqueous solution which is pyrogen-free and has suitable pH, isotonicity and stability. Those of relevant skill in the art are well able to prepare suitable solutions using, for example, isotonic vehicles such as sodium chloride for injection. Preservatives, stabilisers, buffers, antioxidants and/or other additives may be included, as required.

Nasal formulations, for example comprising a thickener such as one of the hydrogels described above may be used. A variety of absorption enhancing agents, mainly surfactants, may also be used for nasal formulations.

The formation may be inserted to into the bladder. Formulations for administration to the bladder are provided. The formulation may comprise the nanofiber of the invention with an aqueous solvent. The formulation may comprise a hydrogel or other gelling agent. The gelling agent may be thermally sensitive and may gel on warming to body temperature, typically 37′C to gel within the bladder and assist in maintaining the formulation within the bladder, for example for the treatment of bladder cancers. Such temperature sensitive gelling agents are generally known in the art. Hydrogels include alginates, chitosans, fibrin actin, silk fibroin, hyaluronic acid, celluloses or mixtures thereof. The formulations may optionally contain a permeation enhancer such as a lipid ester, triglycerides and/or surfactants.

A suitable daily dose can be determined based on age, body weight, administration time, administration method, etc. While the daily doses may vary depending on the condition and body weight of the patient, and the nature of the drug, a typical intravenous dose is about 0.1 mg-2 g/person/day, preferably 0.5-250 mg/person/day.

The invention also provides methods of treating a disease comprising administering a pharmaceutically effective amount of a nanofiber or composition according to the invention.

The compositions or nanofibers according to the invention for use in the treatment of disease are also provided.

Diseases include one or more cancers, schizophrenia, obesity, pain, sleep disorders, psychiatric diseases, neurodegenerative diseases or infectious diseases. Cancers include brain cancers such as glioblastoma multiforme, breast cancer, melanoma, pancreatic cancers, lung cancer, colorectal, bladder, ovarian, endometrial and prostate cancers and ageing. The cancer may be metastatic. Angiotensin 1-7 may be used to treat stroke, neurotensin and other opioid neuropeptides may be used to treat chronic pain, neuropeptide S may be used to treat anxiety, and gastrin releasing peptide may be used, for example, to treat bulimia nervosa and other eating disorders.

The invention also provides methods of producing the nanofibers and compositions according to the invention.

Typically the concentration of the peptide conjugate, prior to nanofiber formation is selected to allow the nanofiber to form polyproline helix upon drying from a solvent. Typically the concentration of GPCR modulator conjugated to the lipophilic moiety, prior to drying to form the nanofiber is at least 120 μM, more typically above 200 μM, 400 μM or above 500 μM or 600 μM, depending on the conjugate used.

The solvent is typically an aqueous solvent, such as a solution of sodium chloride, such as 0.9% w/v) or a dextrose solution, such as 5% w/v

The methods of the invention, for example, include methods of synthesising the nanofibers or compositions of the invention by providing a peptide nanofiber, comprising the peptide GPCR modulator, optionally conjugated to the lipophilic moiety via the selectively cleavable link, and mixing with the drug, optionally in the presence of a solvent, and isolating the nanofiber or compound.

The invention will now be described by way of example only with reference to the following figures.

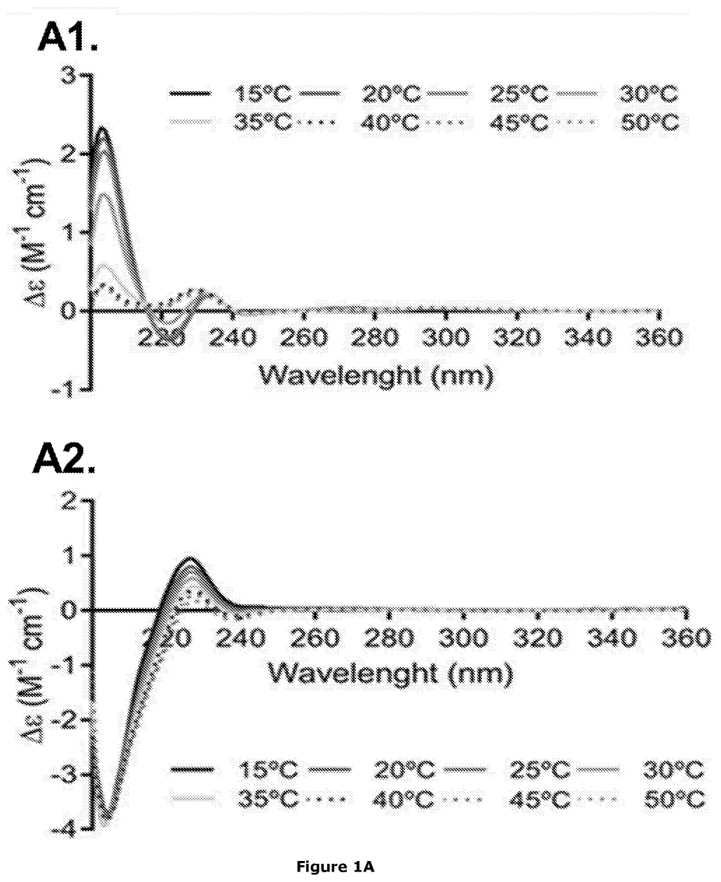

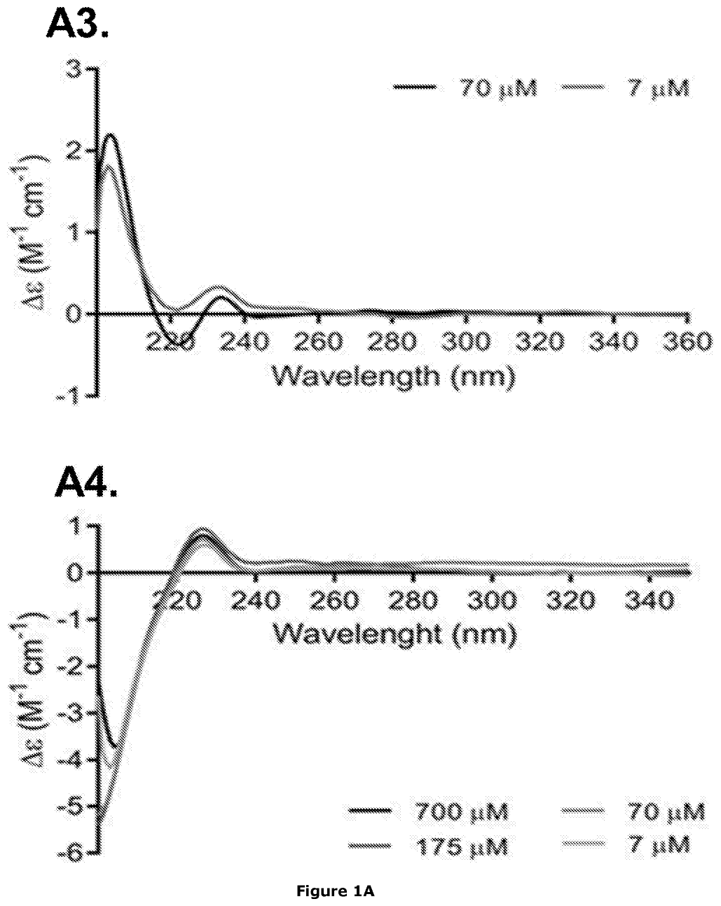

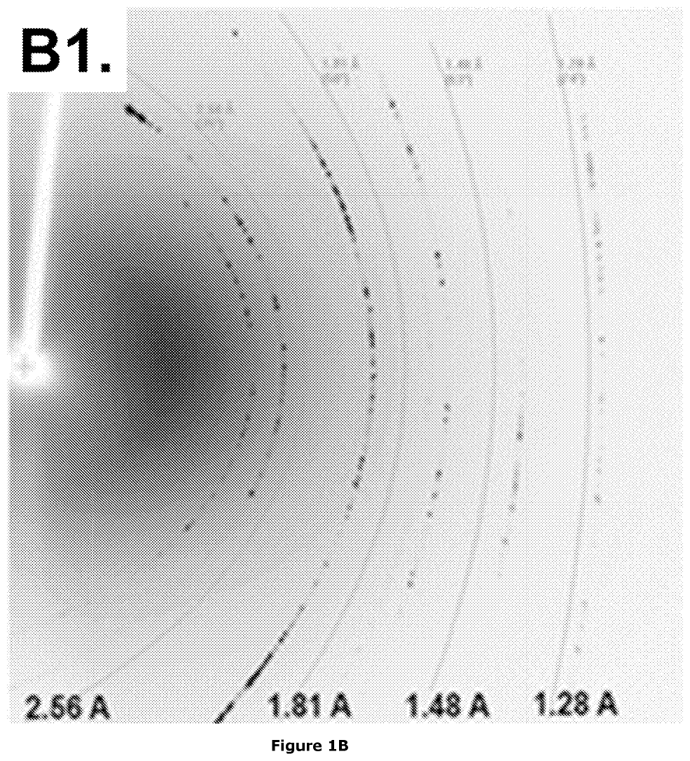

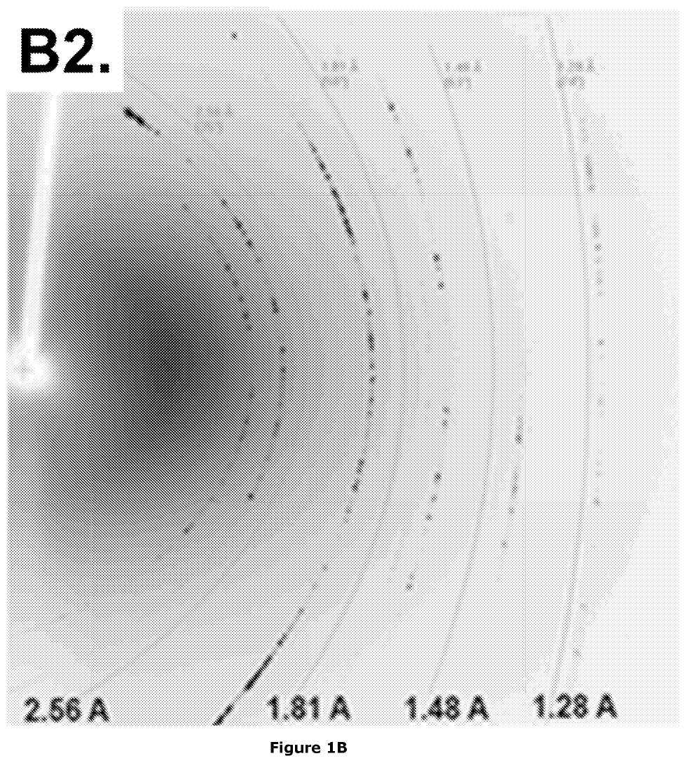

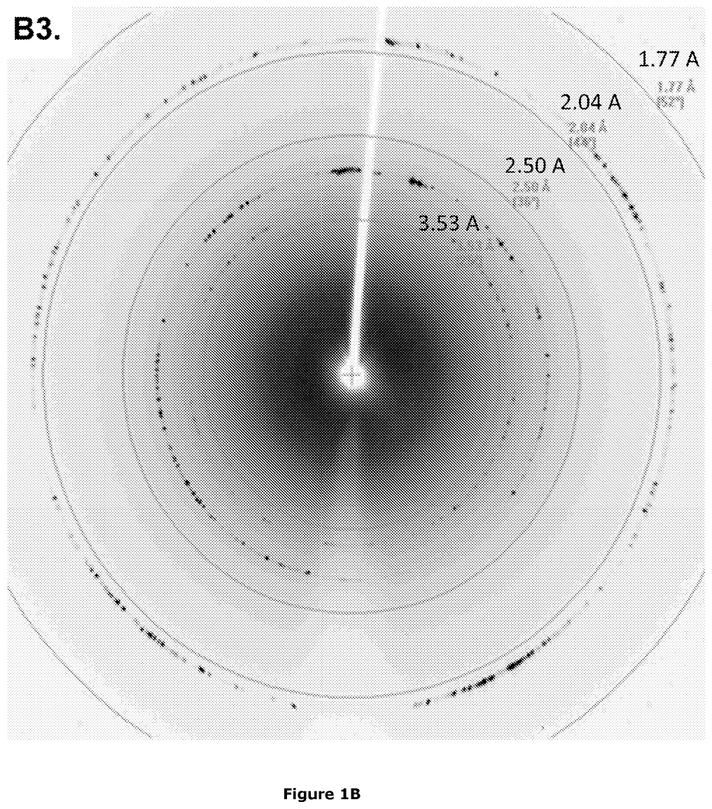

FIG. 1 shows a secondary structure of the TPGnRH. (A) CD spectra of TPGnRH vesicles (A1) and nanofibers (A2) as function of temperature (15-50° C.). Dilution effect on secondary structure of vesicles (A3) and nanofibers (A4) measured at 20° C. (B) XRD of TPGnRH. TPGnRH (B1) vesicles (70 μM) and (B2, B3) nanofibers 700 μM) patterns measured from dried stalks.

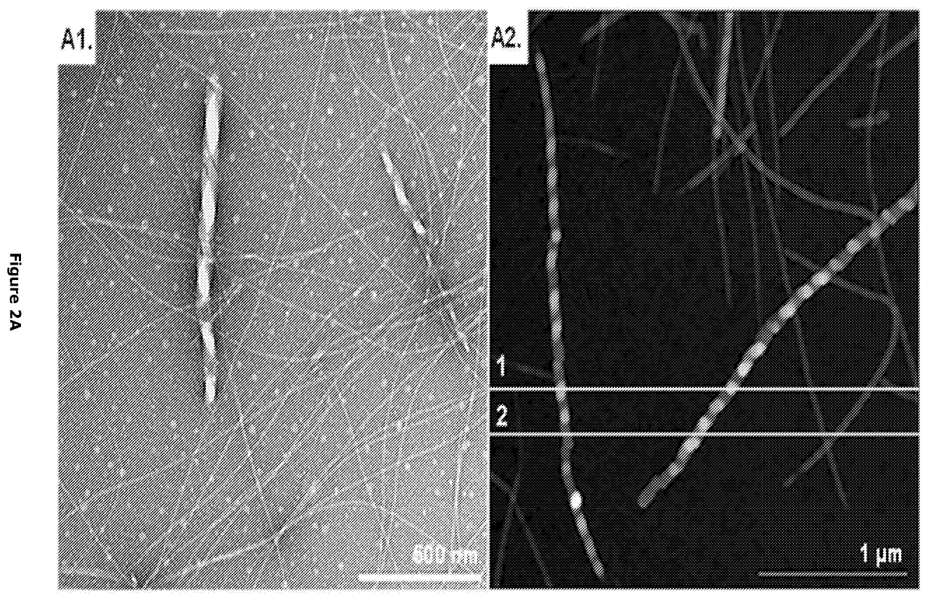

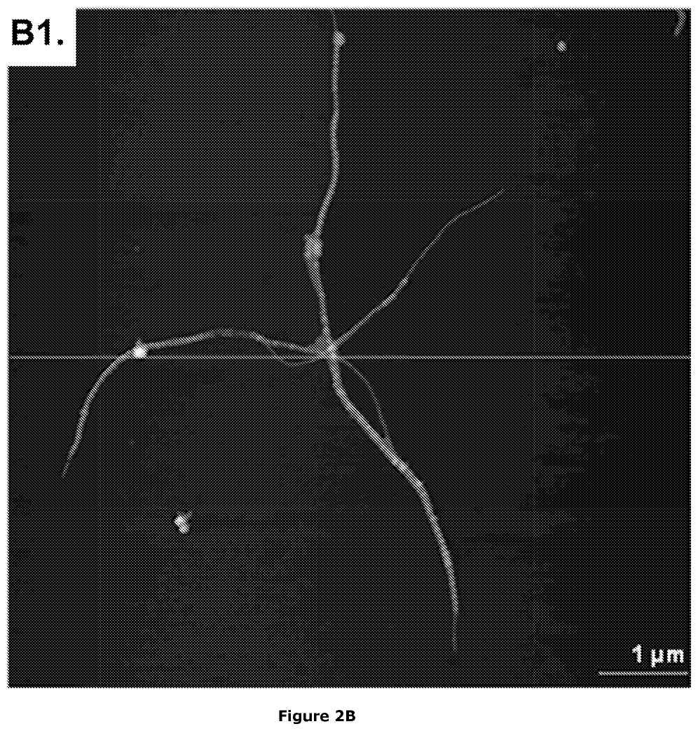

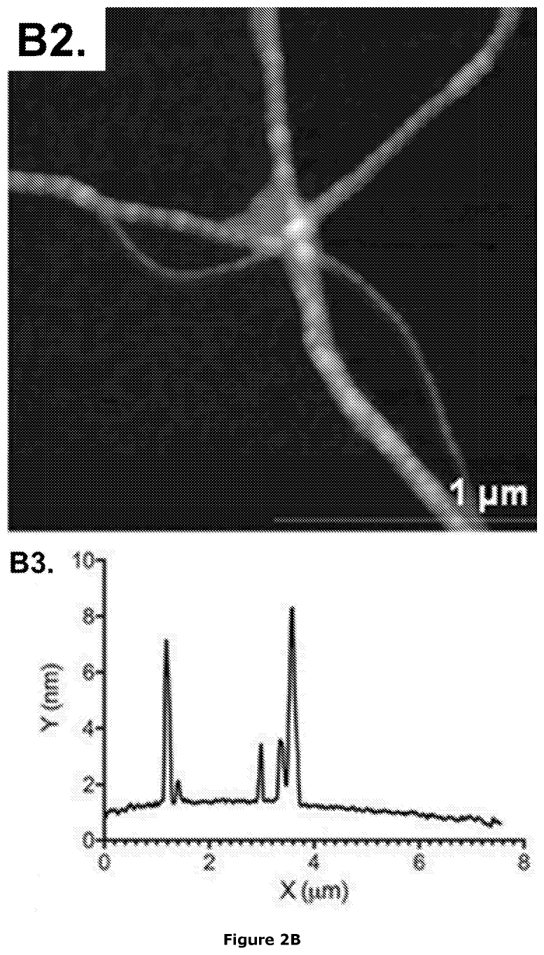

FIG. 2 shows a TEM and AFM images of the TPGnRH nanofibers. (A) TPGnRH aqueous dispersions at 1400 μM analysed by TEM (A1) and AFM (A2). TEM and AFM images confirmed the long axial nanofibers with a twisted ribbon-like morphology. Nanofibers can be present as thin fibres with diameters at 5.72±3.93 nm and heights of 47.67±17.7 nm and thicker twisted ribbon-like nanofibers with diameters between 10.32±2.28 nm to 19.84±0.22 nm (determined by AFM, measurement 1 and 2 showing on A2. (B) AFM images of PAX-loaded nanofibers at 1400 μM.

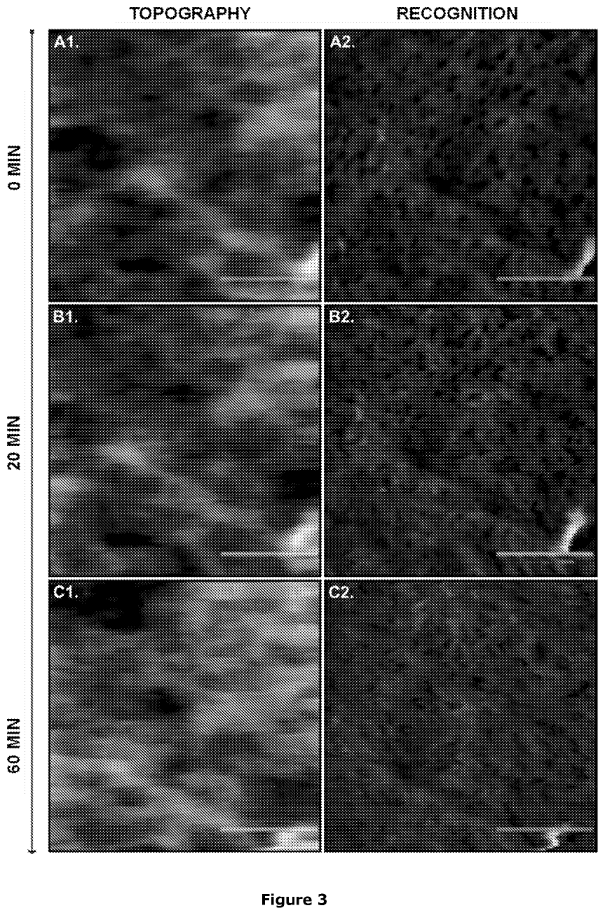

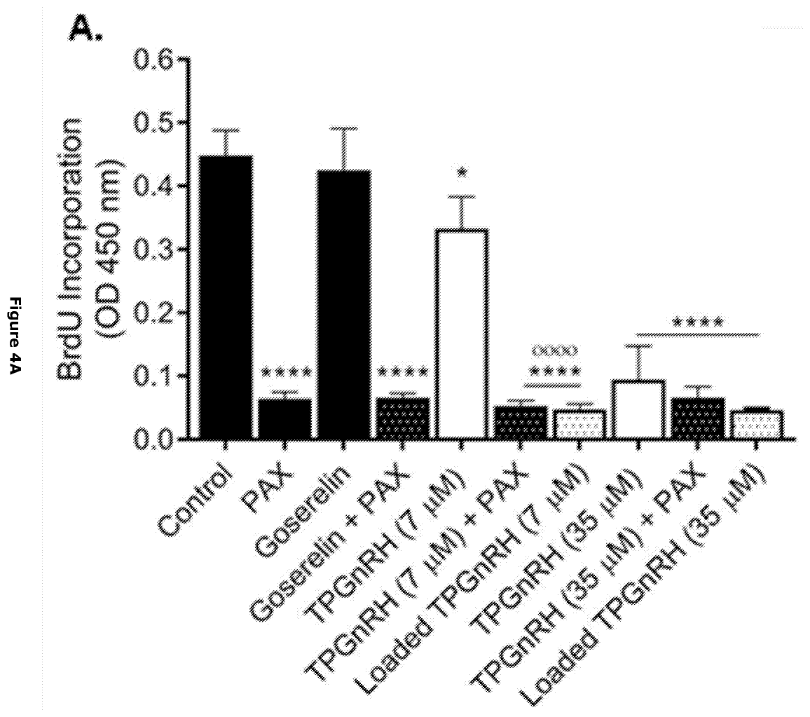

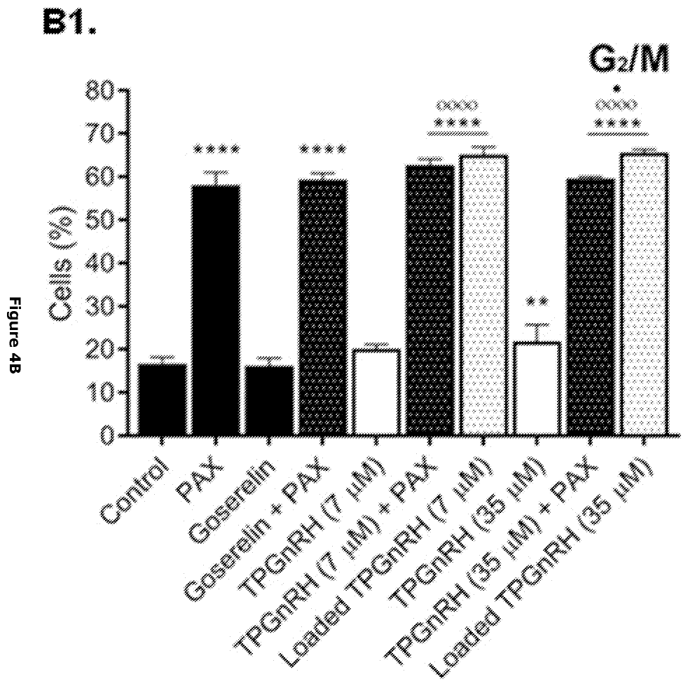

FIG. 3 shows a topographical and recognition images of U-87 MG cells. (A1) Topographical and (B1) recognition image, which shows the binding events (dark spot) corresponding to the amplitude reduction in the maxima of the oscillations due to specific recognition of the TPGnRH nanofibers on the tip to the GnRH-R on the cell surface. (B1, C1) The topography and (B2, C2) recognition images after addition of free TPGnRH nanofibers to block the GnRH-R on the surface of U-87 MG cells. Scale bar 1 μm.

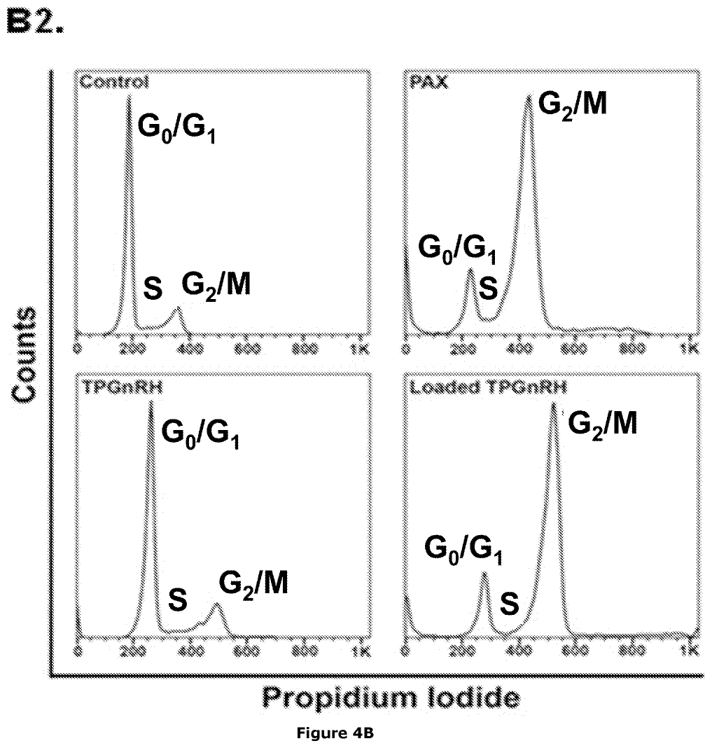

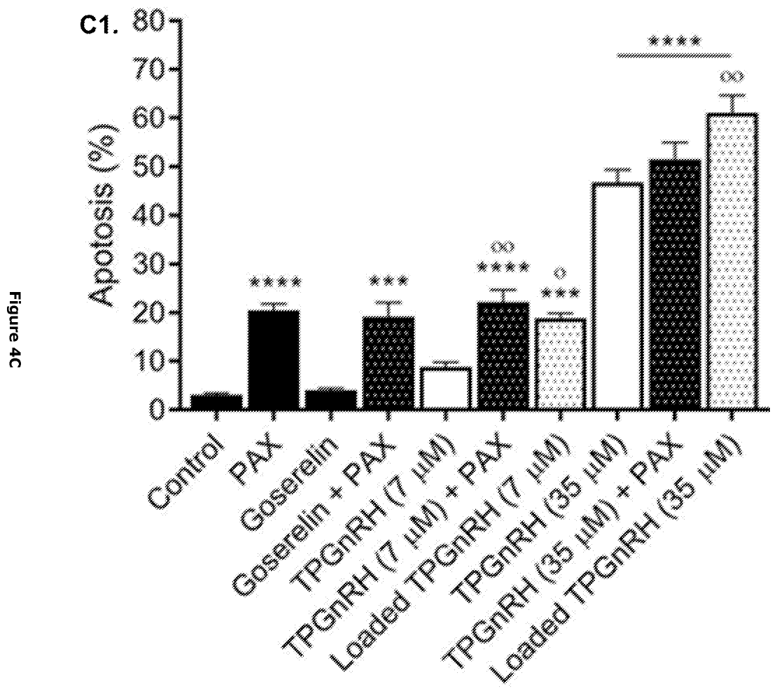

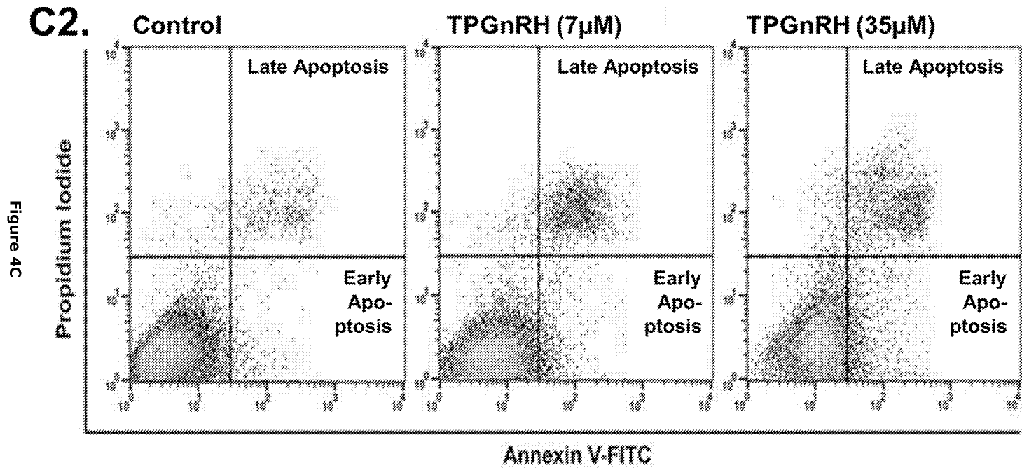

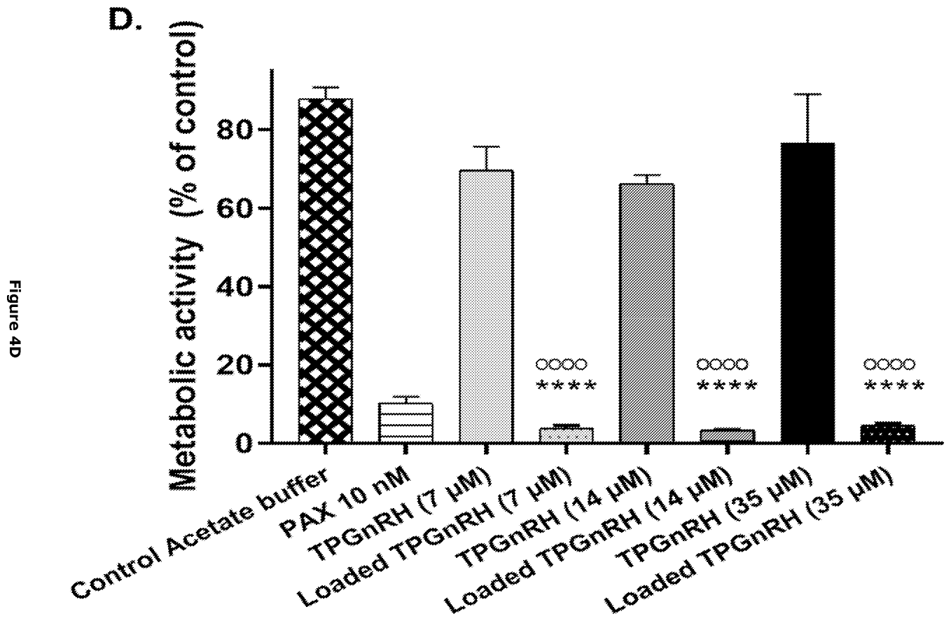

FIG. 4—TPGnRH nanofibers effect on U-87 MG (A) proliferation, (B1, B2) cell cycle, and (C1, C2) apoptosis after 6 days of treatment; (A) * p<0.05, **** p<0.0001 compared to control. ^{∘∘∘∘} p<0.0001 compared to the TPGnRH 7 μM. (B1) * p<0.05, ** p<0.01, **** p<0.0001 compared to control. ^{∘∘∘∘} p<0.0001, compared to cells treated with TPGnRH alone (7 or 35 μM). ⋅ p<0.05 comparing the physical mixture of PAX and TPGnRH at 35 μM with PAX-loaded nanofibers (35 μM of TPGnRH). (B2) Histograms of the cell cycle distribution of control, PAX, TPGnRH 35 μM and PAX-loaded TPGnRH. (C1) *** p<0.01, **** p<0.0001 compared to control. ^{∘} p<0.05, ^{∘∘} p<0.01, compared to the cells treated with TPGnRH alone (7 or 35 μM). (C2) Dot plots showing live cells (negative staining Annexin V-FITC or propidium iodide), early apoptotic (stained for Annexin V-FITC) and late apoptotic (positive for Annexin V-FITC and propidium iodide) cells, and dead cells (only positive for the propidium iodide) at day 6. TPGnRH nanofibers effect on MDA-MB-231 cells (metabolic activity) (D); **** p<0.0001 compared to control. ^{∘∘∘∘} p<0.0001 compared to the TPGnRH 7 μM. One-Way ANOVA with a Tukey's post-hoc. Mean±SD (n=3). Three independent experiments were performed for each set of assays. (D)

### EXAMPLES

### Example 1

### Peptide Synthesis

Synthesis of gonadotropin-releasing hormone (Glu-GnRH) and of a novel lipidic GnRH analogue, Tyrosine (O-Palmitoyl)-Glu-GnRH (TPGnRH), was carried out by standard solid phase methodology using a fluorenylmethyloxycarbonyl (FMOC) chemistry. Peptides were assembled in a rink amide MHBA resin pre-swelled in dimethylformamide (DMF) for 1 hour at room temperature. FMOC orthogonally protected amino acid derivatives (4.2 eq.) were activated with N′N′-diisopropylethylamine (5.0 eq.) and O-(benzotriazol-1-yl)-N,N,N′,N′-tetramethyluronium hexafluorophosphate (HBTU, 4.0 eq.) and coupled twice for 45 minutes each time under stirring. After coupling each residue, Kaiser Test was performed to confirm a complete coupling. Removal of the FMOC was achieved by adding piperidine [20% (v/v) piperidine in DMF, ˜10 mL] to the resin beads under agitation for 10 minutes. Deprotection step was repeated twice to ensure a complete removal, and Kaiser Test was performed to ensure the presence of free amines. Once peptide synthesis was complete, the resin was washed with copious amounts of DMF followed by a mixture of dichloromethane and methanol (1:1). Resin was dried under vacuum, and transferred into a pre-weighed glass vial and stored in a silica desiccator overnight before the cleavage of the peptide from the resin. Cleavage of the peptides from the resin was achieved with a mixture of trifluoroacetic acid, triisopropylsilane, water (TFA:TIS:H₂O, 95:2.5:2.5 v/v) for 4 hours, under stirring, at room temperature. The reaction mixture was evaporated under vacuum using a liquid nitrogen trap for 4 hours. The peptide was precipitated with frozen (−20°) diethyl ether, and the precipitate was collected by centrifugation (1,200 rpm for 15 minutes, twice). Pellet was re-dissolved in water and freeze-dried.

The esterification of the free phenolic hydroxyl group of Tyrosines of Glu-GnRH was obtained by attaching a palmitic tail. Tyr-Gly-Leu-Arg-Pro-Gly peptide was synthesised as above. The 2-chlorotityl protecting group of tyrosyl residue was cleaved by reacting the dried resin with a mixture of dichloromethane, trifluoroacetic acid, triisopropylsilane (DCM:TFA:TIS, 90:5:5 v/v) for 10 minutes under stirring. The latter step was repeated four times. Beads were washed with copious amounts of DMF and swelled for one hour in DMF. The N-hydroxysuccinimide ester of palmitic acid (8.0 eq.) in ˜5 mL of DMF and triethylamine (16.1 eq.) were added to the resin in DMF and reacted for 24 hours under stirring at room temperature. Once the reaction was completed, resin was washed with copious amounts of DMF and remaining synthesis was continued as described above. Palmitoyl GnRH (TPGnRH) was synthesised by grafting a palmitic tail (C16) to the free phenolic hydroxyl group of Tyr⁵ via an ester bond allowing for the in vivo conversion of the lipidised peptide into the Glu-GnRH by esterases.

### Example 2

### Peptide Purification

Crude peptides were purified using solid phase extraction (Sep-Pak plus C18 column, Waters). Initially, a column was equilibrated with acetonitrile (ACN, 5 mL) and washed with a mixture of trifluoroacetic acid and water (TFA:H₂O, 1:999 v/v). Peptide (15 to 20 mg) was dissolved in 1 M hydrochloric acid (HCl, 2 mL) and loaded into the column. Following to the peptide loading, the column was washed with TFA:H₂O (10 mL), 1 M HCl (10 mL) and de-ionised water (5 mL). Elution of the peptide fractions was achieved by washing the column with 5% (v/v) ACN in water (2 mL), 45% (v/v) of ACN (2 mL), 65% (v/v) ACN (6 mL), and 100% (v/v) ACN (4 mL), sequentially. The Glu-GnRH was eluted between 5 to 45% of ACN and the TPGnRH was eluted at >45% of ACN. Purity of the peptide fractions was quantified by using reverse-phase high performance liquid chromatography (RP-HPLC). Analysis was carried out on Onyx Monolithic C18 column (4.6 mm×10+100 nm, 5 μm) with the gradient method (Table 1) using an Agilent 1100 Series HPLC system (Agilent Technologies, Cheadle, UK). Flow rate 1.5 mL min⁻¹ at 25° C. Injection volume was 10 μL and detection was performed at 220 and 280 nm. The retention time was 9.7 minutes for Glu-GnRH and 22.0 minutes for TPGnRH.

TABLE 1
RP-HPLC gradient method for GnRH peptides.

| Time | A: 0.1% (v/v) | B: 0.08% (v/v) |
|---|---|---|
| (minutes) | TFA in H₂O | TFA in ACN |
| 0 | 90 | 10 |
| 5 | 90 | 10 |
| 15 | 50 | 50 |
| 18 | 50 | 50 |
| 28 | 40 | 60 |
| 33 | 20 | 80 |
| 38 | 90 | 10 |

The fractions containing peptide with purity above 95% were dried in a Savant ISS110 SPEEDVAC (Thermo Scientific, Paisley, UK) or diluted with de-ionised water [<10% (v/v) ACN] and freeze-dried.

### Example 3

### Peptide Characterisation

Both peptides were characterised by electrospray ionisation (positive/negative) mass spectrometry (MS), nuclear magnetic resonance (NMR), RP-HPLC, attenuated total reflectance Fourier-transformed infrared (ATR-FTIR) spectroscopy, thermogravimetric analysis (TGA) and differential scanning calorimetry (DSC). The isoelectric point of the peptides was also determined using an acid-base titration.

MS exhibited the m/z of 1437.56 Da in agreement with the calculated mass (1438.73 Da) and NMR illustrated the peaks corresponding to protons of the methylene and methyl groups of the palmitic moiety [¹H NMR in H₂O:D₂O (90:10), 15 mM, 600 MHz, δ (ppm): 0.78 (CH₃) and 1.20 (CH₂)] and the characteristic peaks of the GnRH. FTIR spectroscopy showed two strong bands at 2924 and 2852 cm⁻¹ corresponding to the vibration of the palmitoyl moiety (CH stretch) and a peak at 1635 cm⁻¹ confirming the presence of an ester bond between the Tyrosines and the palmitic tail (C═O stretch). Isoelectric point of the TPGnRH is 6.28±0.51, and TGA and MTDSC showed the enhanced thermal stability of TPGnRH in comparison to Glu-GnRH. Glass transition temperature of TPGnRH is 103.4° C.

### Example 4

### Critical Aggregation Studies

Critical aggregation concentration (CAC) of TPGnRH was determined using pyrene ¹⁻⁴ and thioflavin T ^{5, 6}. The peptide solutions (3.5-700 μM) were prepared in phosphate-buffered saline (PBS, 0.01 M, pH 7.4) either by a dilution of a stock solution at 700 μM or solutions prepared at the required concentration. In the pyrene assay, pyrene was dissolved in methanol at 250 μM, dispersed into a black 96-well plate, and methanol was evaporated overnight. TPGnRH solutions were added to pyrene and incubated at 37° C. for 24 hours under stirring. Fluorescence spectra was recorded λₑₓ at 334 nm. For the thioflavin T assay, peptide solutions were added to freshly prepared thioflavin Tat 50 μM. All peptide samples were incubated during 5 minutes at room temperature and fluorescence was measured at λₑₓ at 450 nm and λₑₘ at 482 nm.

The lipidisation of the Glu-GnRH enabled the peptide to self-assemble into nanofibers. Pyrene assay showed the ability of TPGnRH to self-assemble into aggregates at CAC of 7 μM, while thioflavin T suggested that at a minimal CAC of 135 μM, TPGnRH self-assembles into long-axial nanofibers.

### Example 5

### Secondary Structure

Secondary structure was assessed using circular dichroism (CD). TPGnRH solutions (70 or 700 μM) were prepared in aqueous 0.9% sodium chloride and analysed at 20±0.1° C. using Applied Photophysics Pi180 spectropolarimeter (Surrey, UK). For the temperature-dependent studies, the peptide was incubated at a range of temperature (15 to 50° C.) and the spectra were collected at 5° C. intervals. To investigate the effect of dilution, peptide solutions were diluted in 0.9% (w/v) sodium chloride solution at 20° C. Data were collected every 1 nm over the wavelength range of 200-360 nm. X-ray diffraction (XRD) studies were carried out on stalks prepared by suspending drops of TPGnRH solutions (70 or 700 μM) between the ends of the wax-coated capillaries and allowing them to dry at room temperature. Stalks were vertically mounted onto a four-axis goniometer of a FR-E+X-Ray diffractomer (Rigaku, Sevenoaks, UK).

At a low concentration (70 μM), the TPGnRH assumes a β-turn type II as a secondary structure, however when assembled as nanofiber TPGnRH arranges into poly (proline) type II (PPII) helix. CD studies at the range of temperatures (15 to 50° C.) and dilutions (1:10-1:100) showed the physical stability of the nanofibers at temperatures <40° C. and upon dilution. XRD patterns revealed a pseudo-crystalline structure in the fibres, and partially amorphous structure at the low concentration (70 μM) with similar peaks. FIG. 1 shows the secondary structure of TPGnRH (70 and 700 μM) as a function of the temperature and dilution illustrating the stability of the nanofibers in comparison to the vesicles. FIG. 1 also shows the XRD patterns of TPGnRH.

### Example 6

### Preparation of Self-Assembled Nanofibers

The self-assembled TPGnRH nanofibers were prepared by dissolving the dry peptide in water or phosphate-buffered solution (0.01 M, pH 7.4) followed by vortex and a bath sonication. Morphology was studied by adding a drop of the TPGnRH solutions on the coated-side of a copper grid (Formvar/Carbon coated grid, F196/100 3.05 mm, Mesh 300, TAAB, UK) and stained with 2% (w/v) uranyl acetate. The grids were blotted using Whatman N01 filter paper. Imaging was carried out using a JEM-1400 Transmission Electron Microscope (Jeol, Herts, UK). The morphology was also studied using atomic force microscopy (AFM). A drop of TPGnRH was placed onto cleaved muscovite mica and imaged with a silicon probe with resonance frequency of 288 to 388 KHz, spring constant 12 to 103 N m⁻¹, 140-180 μm length and tip curvature 3.6-5.6 μm. All images were acquired with Multi Mode/NanoScope IV scanning probe microscope (Digital Instruments, Santa Barbara, USA) using Tapping Mode.

Morphological investigations demonstrated that TPGnRH forms ribbon-like nanofibers with a diameter of 5.72±3.93 to 19.84±0.22 nm determined by TEM and AFM.

### Example 7

### Preparation of Self-Assembled Nanofibers Loaded with Paclitaxel

Paclitaxel (PAX)-loaded TPGnRH nanofibers were prepared by adding a PAX solution (250 μL, 2.562 mg mL⁻¹ in isopropyl alcohol, IPA) to the dry peptide to achieve a molar ratio of 1:1, 1:1.95, 1:3.68 or 1:7.36 of PAX:TPGnRH. After vortexing, bath and probe sonication (200 watts, amplitude 60%, UP200S Ultrasonic, SciMed, Stockport, UK) for 10 minutes, the IPA was removed by rotary evaporation. The film was re-hydrated in acetate buffer (50 mM, pH 4.5), and the final formulation was centrifuged for 5 minutes at 900 rpm to remove undissolved PAX. Supernatant was collected and the amount of PAX and TPGnRH was quantified by reverse phase-HPLC. Analysis was conducted on Onyx Monolithic C18 column (4.6 mm×10+100 nm, 5 μm pore size) with a gradient method (Table 2) using an Agilent 1100 Series HPLC system (Agilent Technologies, Cheadle, UK). The PAX was eluted with a flow rate of 1.5 mL min⁻¹ at 25° C. Injection volume was 40 μL, and the detection was performed at 227 nm. The time of retention was 7.32 minutes for PAX and 13.90 minutes for TPGnRH. Morphology of PAX-loaded formulations was evaluated using TEM and AFM, as described above.

TABLE 2
Reverse phase-HPLC gradient method for PAX and TPGnRH.

| | A: 0.1% (v/v) | B: 0.08% (v/v) |
|---|---|---|
| Time (minutes) | TFA in H₂O | TFA in ACN |
| 0 | 70 | 30 |
| 7.5 | 50 | 50 |
| 10.5 | 50 | 50 |
| 20.5 | 40 | 60 |
| 26.5 | 6 | 94 |
| 29.5 | 70 | 30 |

Encapsulation of PAX was achieved using a solvent evaporation method with constant amount of PAX (3 μmol) and increasing amounts of TPGnRH. Encapsulation efficiency reached 68.67±3.59% at the PAX:TPGnRH molar ratio of 1:7.36. At this molar ratio, the aqueous solubility of PAX is 1759.21±91.98 μg mL⁻¹, which is ˜1900-fold higher than the aqueous solubility of PAX in aqueous buffer solution (0.92±1.35 μg mL⁻¹) (Table 3). TEM images show the presence of thin and short nanofibers with a diameter of 9.48±2.54 nm and length of 199.96±46.05 nm, which are associated with vesicles of 5.16±0.66 nm. AFM images further confirmed the ribbon-like structure of the PAX-loaded TPGnRH with a diameter of 3.74±2.15 nm.

TABLE 3
Loading and encapsulation efficiencies
of PAX-loaded TPGnRH nanofibers.

| PAX:TPGnRH | Loading Efficiency | Encapsulation |
|---|---|---|
| Molar Ratio | (% w/w) | Efficiency |
| 1:1 | 13.06 ± 4.33 | 19.30 ± 7.71 |
| 1:1.95 | 16.26 ± 2.31 | 50.67 ± 7.50 |
| 1:3.68 | 7.43 ± 1.35 | 54.61 ± 5.69 |
| 1:7.36 | 5.52 ± 0.14 | 68.67 ± 3.59 |

FIG. 2 shows the TEM and AFM images of unloaded and PAX-loaded nanofibers.

### Example 8

### Critical Aggregation Studies with Paclitaxel-Loaded Nanofibers

PAX:TPGnRH formulation at a molar ratio 1:7.36 was diluted in PBS (0.01 M, pH 7.4) from 22,000 to 7 μM of TPGnRH. Diluted formulations (20 μL) were added to 80 μL of Thioflavin T at 50 μM, incubated for 5 minutes at room temperature, and fluorescence was measured at λex 450 and λem 482 in a black 96-well plate using the Synergy H1 Microplate Reader (BioteK, Vermont, USA).

The immobilisation of thioflavin T within the PAX-loaded nanofibers is reflected by the increase of fluorescence intensity at a CAC of 2.52 mM (3.6 mg mL⁻¹).

### Example 9

### Release Studies of PAX-Loaded TPGnRH Nanofibers

The release studies were performed by entrapping PAX-loaded nanofibers in a dialysis membrane with the molecular weight cut-off (MWCO) of 1,000 Da (Spectra/Por® 7 Dialysis Membrane, USA) and assessing the amount of drug released out of the bag. PAX-loaded nanofibers at 1:7.36 ratio (230 μL, 392.76 μg of PAX) were added to the dialysis bag and dialysed against 1% (w/v) Soluplus® in PBS (50 mL) to ensure sink conditions in a shaking water bath (Kottermann 3047, Kottermann Ltd., Wooburn Green, UK) at 37° C. At regular intervals, 500 μL of release medium was collected and the amount of PAX was quantified using RP-HPLC with the gradient method described above (Table 3.2, Section 3.2.2.2).

Due to the low solubility of PAX in PBS buffer (0.92±1.35 μg mL⁻¹), the release assay was performed in Soluplus® (1% w/v) in PBS, in which PAX solubility is 29.30±3.63 μg mL⁻¹ and ensuring the volume is large enough to ensure sink conditions. A burst release of PAX from TPGnRH nanofibers was observed during the first 8 hours (40%) followed by a gradual near-complete release until 72 hours. After the 3 days, a plateau was observed with 76.11±3.10% of PAX released in the in vitro studies.

### Example 10

### Physical and Chemical Stability of PAX-Loaded TPGnRH Nanofibers

The PAX-loaded TPGnRH (1:7.36 PAX:TPGnRH) were freshly prepared as previously described, and 50 μL were added to 250 μL of 5% (w/v) dextrose solution in a glass HPLC vial (1 cm width and 3 cm height). Vials were freeze-dried for 24 hours under vacuum in Edwards Modulyo® Freeze Dryer (Thermo Scientific, Paisley, UK) with a freeze-drier pump system 320015 (Gardner Denver Ltd., Medstead, UK) and stored at −20° C. At specific time-points, samples were re-suspended in de-ionised water (300 μL) and analysed by RP-HPLC to quantify the amount of PAX and TPGnRH. For the RP-HPLC analysis, the formulation (20 μL) was added to a mixture of ACN and H₂O (50:50, 980 μL), and analysed using the gradient method described on Table 2. The samples were imaged in the TEM to assess their morphology by diluting the sample (10 μL) in water (90 μL) and negatively stained as described above.

Freeze-dried formulations presented as a strong and porous freeze-dried cake and no changes were observed in the height of the cake over 12 months. No differences were observed in the content of both drugs (PAX, TPGnRH) over 12 months of cold storage. All formulations showed the presence of nanofibers with no alterations in morphology.

### Example 11

### Enzymatic Stability

Fresh blood, obtained from adult male Wistar rats, was collected in sterile the 170 I.U. lithium heparin-coated tubes (Vacutainer, BD Bioscience, UK). Blood was centrifuged immediately at 2,000 g for 15 minutes (4° C.) and supernatant was collected and diluted to 50% (v/v) with PBS. Livers and brains were obtained from adult male Wistar rats, and the tissues were blotted to dryness, weighed, sliced into pieces, and homogenised with ice-cold PBS (in 2 mL of buffer g⁻¹ of tissue) using the glass homogeniser. Then, tissue homogenates were centrifuged at 4,300 g for 1 hour (4° C.) and the supernatants collected. Glioblastoma cell homogenates were prepared using U-87 MG cells. Cells were cultured in Eagle's Minimum Essential Medium (EMEM) supplemented with 10% (v/v) heat-inactivated fetal bovine serum (FBS), 1% (v/v) of non-essential amino acids (NEAA) and 1 mM of sodium pyruvate in T75 flasks with a surface area of 75 cm² and maintained in a humidified atmosphere of 5% CO₂ at 37° C. Breast cancer cell homogenates were prepared using MDA-MB-231 cells. Cells were cultured in Dulbecco's Modified Eagle Medium (DMEM) supplemented with high levels of glucose (4.5 g/L), 10% (v/v) heat-inactivated fetal bovine serum (FBS), 1% (v/v) of non-essential amino acids (NEAA) and penicillin and streptomycin (500 units/mL) in T75 flasks with a surface area of 75 cm² and maintained in a humidified atmosphere of 5% CO₂ at 37° C. Media was changed for both cell lines every 3 to 4 days. After reaching confluence, cells were washed twice with Hank's Balanced Salt Solution (HBSS) and incubated with ˜2 mL of TrypLE Enzyme at 37° C. for ˜3 minutes. Then, complete media were added, and cells were centrifuged at 1,000 rpm for 5 minutes in C-28A centrifuge (Boeco, Germany). U-87 MG or MDA-MB-231 cells (˜10×10⁶ cells mL⁻¹) were re-suspended in ice-cold PBS, and homogenised with a 2×5 second pulses with the probe sonicator (UP200S Ultrasonic processor, SciMed, 200 watts, amplitude of 60%). Cell debris were removed by centrifugation at 2,000 rpm for 5 minutes (Jouan B4i, Thermo Scientific, Paisley, UK). Supernatant was collected and stored at −80° C. Total protein concentration in the homogenates was determined using the Bradford assay with bovine serum albumin as a standard. Diluted plasma, tissue or cell homogenates were pre-warmed during ˜30 minutes at 37° C. Peptide stock solutions (Glu-GnRH, goserelin acetate and TPGnRH, 5 mM in PBS, 500 μL) were prepared and added to the homogenate solutions (120 μL). PAX-loaded nanofibers prepared at a ratio of 1:7.36 [PAX:TPGnRH consisting of 3 μmol (2.56 mg mL⁻¹) of PAX, 22.02 μmol (31.46 mg mL⁻¹) of TPGnRH], as previously described, and further diluted in PBS to achieve a concentration of 5 mM of TPGnRH and 500 nM of PAX (120 μL) were added to the homogenates. At various time intervals, an aliquot was collected (80 μL) and added to ice-cold ACN (80 μL) to quench the enzymatic activity. Samples were stored at −80° C. for at least 3 hours to maximise protein precipitation. Samples were vortexed for 15 minutes, centrifuged for 15 minutes at 12,000 rpm, and the supernatants were collected and analysed by RP-HPLC. RP-HPLC analysis was conducted as described above using a gradient method with a mobile phase consisting of a mixture of 0.1% (v/v) TFA in water and 0.08% (v/v) TFA in ACN mixed at ratio described as Table 2 for PAX-loaded TPGnRH and Table 1 for Glu-GnRH, goserelin and TPGnRH.

TPGnRH nanofibers (unloaded or loaded with PAX) exhibited an excellent enzymatic stability in biological media [50% (v/v) rat plasma, brain and liver homogenates and in glioblastoma cell homogenate] comparing to the parent peptide, Glu-GnRH. In plasma and brain homogenates, nanofibers remained stable along 8 hours (>80% of peptide remaining), while in liver homogenates TPGnRH follows a first-order two-phase decay with a fast degradation within the first hour followed by a slow degradation with ˜50% of the remaining peptide after 8 hours. Tables 4, 5, 6, and 7 illustrate the degradation kinetics of TPGnRH and PAX-loaded TPGnRH in 50% (v/v) plasma, brain, liver, and U-87 MG homogenates, respectively.

TABLE 4
Degradation kinetics in 50% (v/v) rat plasma homogenates.

| | | | | | | |
|---|---|---|---|---|---|---|
| | C₀ | | Plateau | | | AUC₀₋₁₂₀ |
| t_{1/2} | (μg | k | (μg | | | (μg mL⁻¹ |
| (minutes) | mL⁻¹) | (min⁻¹) | mL⁻¹) | Equation | r² | min⁻¹) |

Glu-GnRH

| | | | | | | |
|---|---|---|---|---|---|---|
| 17.66 ± 4.22 | 103.6 | 0.04021 | 3.952 | First-Order | 0.9919 | 2817 ± 210.3 |
| | | | | C = 99.7e^{-0.04t + 3.95} | | |

TPGnRH

| | | | | | | |
|---|---|---|---|---|---|---|
| 445.14 ± 4.95 | 83.33 | 0.0937 | — | Zero-Order | 0.5201 | 9294 ± 200.1 |
| | | | | C = 83.3-0.094t | | |

PAX-Loaded TPGnRH (TPGnRH)

| | | | | | | |
|---|---|---|---|---|---|---|
| 647.40 ± 6.95 | 647.4 | 1.000 | — | Zero-Order | 0.6456 | 7067 ± 126.8 |
| | | | | C = 647.4-1.00t | | |

PAX-Loaded TPGnRH (PAX)

| | | | | | | |
|---|---|---|---|---|---|---|
| 266.17 ± 4.51 | 66.33 | 0.1246 | — | Zero-Order | 0.5075 | 7148 ± 193.0 |
| | | | | C = 66.3-0.125t | | |

TABLE 5
Degradation kinetics in 50% (v/v) rat brain homogenates.

| | | | | | | |
|---|---|---|---|---|---|---|
| | C₀ | | Plateau | | | AUC₀₋₄₈₀ |
| T_{1/2} | (μg | k | (μg | | | (μg mL⁻¹ |
| (minutes) | mL⁻¹) | (min⁻¹) | mL⁻¹) | Equation | r² | min⁻¹) |

Glu-GnRH

| | | | | | | |
|---|---|---|---|---|---|---|
| 3.59 ± 0.08 | 503.0 | 0.1928 | 3.952 | First-Order | 0.9966 | 3251 ± 54.58 |
| | | | | C = 495.0 e^{-0.19t + 8.03} | | |

TPGnRH

| | | | | | | |
|---|---|---|---|---|---|---|
| 1951.40 ± 8.01 | 694.7 | 0.1781 | — | Zero-Order | 0.7566 | 313369.7 ± 2418.77 |
| | | | | C = 694.7-0.178t | | |

PAX-Loaded TPGnRH (TPGnRH)

| | | | | | | |
|---|---|---|---|---|---|---|
| 784.91 ± 4.10 | 671.1 | 0.4275 | — | Zero-Order | 0.9221 | 271656.7 ± 2202.42 |
| | | | | C = 671.1-0.428t | | |

PAX-Loaded TPGnRH (PAX)

| | | | | | | |
|---|---|---|---|---|---|---|
| 931.51 ± 6.57 | 66.51 | 0.0357 | — | Zero-Order | 0.8148 | 27041.67 ± 160.24 |
| | | | | C = 66.5-0.036t | | |

TABLE 6
Degradation kinetics in 50% (v/v) rat liver homogenates.

| | | | | | | | | |
|---|---|---|---|---|---|---|---|---|
| | | C₀ | | Plateau | | | | AUC₀₋₄₈₀ |
| T_{1/2α} | T_{1/2β} | (μg | k_{α} | k_{β} | (μg | | | (μg mL⁻¹ |
| (minutes) | (minutes) | mL⁻¹) | (min⁻¹) | (min⁻¹) | mL⁻¹) | Equation | r² | min⁻¹) |

Glu-GnRH

| | | | | | | | | |
|---|---|---|---|---|---|---|---|---|
| 0.67 ± 0.03 | — | 566.5 | 1.088 | — | 16.58 | First-Order (One-Phase Decay) | 0.9901 | 855.9 ± 49.78 |
| | | | | | | C = 94.9 e^{-1.09t + 16.58} | | |

TPGnRH

| | | | | | | | | |
|---|---|---|---|---|---|---|---|---|
| 4.94 ± 2.61 | 196.47 ± 84.90 | 863.8 | 0.1678 | 0.005339 | 359.3 | First-Order (Two-Phase Decay) | 0.9868 | 203321 ± 2344 |
| | | | | | | C = 359.3 + 3408.9 e^{-0.17t} + 1636.09 e^{-0.01t} | | |

PAX-Loaded TPGnRH (TPGnRH)

| | | | | | | | | |
|---|---|---|---|---|---|---|---|---|
| 42.33 ± 2.61 | 54.81 ± 0.01 | 605.8 | 0.0163 | 0.01265 | 311.0 | First-Order (Two-Phase Decay) | 0.9338 | 173268 ± 2180 |
| | | | | | | C = 311.0 + 9.78 × 10⁻⁹ e^{-0.02t} + 294.8 e^{-0.01t} | | |

PAX-Loaded TPGnRH (PAX)

| | | | | | | | | |
|---|---|---|---|---|---|---|---|---|
| 1.52 ± 0.50 | 78.02 ± 8.08 | 80.7 | 0.3255 | 0.0101 | 38.57 | First-Order (Two-Phase Decay) | 0.9516 | 21714 ± 169.6 |
| | | | | | | C = 38.6 + 130.3 e^{-0.33t} + 290.9 e^{-0.01t} | | |

TABLE 7
Degradation kinetics in U-87 MG cell homogenates.

| | | | | | | |
|---|---|---|---|---|---|---|
| | C₀ | | Plateau | | | AUC₀₋₄₈₀ |
| T_{1/2} | (μg | k | (μg | | | (μg mL⁻¹ |
| (minutes) | mL⁻¹) | (min⁻¹) | mL⁻¹) | Equation | r² | min⁻¹) |

Glu-GnRH

| | | | | | | |
|---|---|---|---|---|---|---|
| 3.59 ± 0.08 | 503.0 | 0.1928 | 3.952 | First-Order | 0.9966 | 3251 ± 54.58 |
| | | | | C = 495.0 e^{-0.19t + 8.03} | | |

TPGnRH

| | | | | | | |
|---|---|---|---|---|---|---|
| 1951.40 ± 8.01 | 694.7 | 0.1781 | — | Zero-Order | 0.7566 | 313369.7 ± 2418.77 |
| | | | | C = 694.7-0.178t | | |

PAX-Loaded TPGnRH (TPGnRH)

| | | | | | | |
|---|---|---|---|---|---|---|
| 784.91 ± 4.10 | 671.1 | 0.4275 | — | Zero-Order | 0.9221 | 271656.7 ± 2202.42 |
| | | | | C = 671.1-0.428t | | |

PAX-Loaded TPGnRH (PAX)

| | | | | | | |
|---|---|---|---|---|---|---|
| 931.51 ± 6.57 | 66.51 | 0.0357 | — | Zero-Order | 0.8148 | 27041.67 ± 160.24 |
| | | | | C = 66.5-0.036t | | |

MDA-MB-231 cell homogenates
Glu-GnRH

| | | | | | | |
|---|---|---|---|---|---|---|
| 4.47 ± 1.27 | 1422 | 0.1551 | 552.9 | First-Order | 0.6382 | 1202193 ± 51803 |
| | | | | C = 1422 e-0.1551t | | |

TPGnRH

| | | | | | | |
|---|---|---|---|---|---|---|
| 3117 ± 524 | 709.5 | 2.224 | 325.8 | Zero-Order | 0.6375 | 6279033 ± 413474 |
| | | 10⁻⁴ | | C = 709.5-0.0002224t | | |

### Example 12

### Haemolytic Toxicity

Fresh blood, obtained from adult male Wistar rats (250 to 300 g) (Bioresources Unit, School of Pharmacy and Biomedical Sciences, University of Portsmouth, Portsmouth, UK) was collected in sterile lithium heparin-coated tubes (170 I.U., Vacutainer, BD Bioscience, UK) and kept on ice. To isolate the rat red blood cells (RBCs), fresh blood was centrifuged at 2,000 g using Haraeus Multifuge 3 S-R (Thermo Scientific, UK) at 4° C. for 10 minutes. The supernatant was removed, and the final volume was raised to the whole blood volume with PBS (0.01 M, pH 7.4). RBCs were centrifuged at 2,000 g for 10 minutes and washed with PBS three times. After the washing steps, two cell suspensions were prepared: 4% (w/v) RBCs in PBS and 1% (v/v) Triton X-100 (i.e. 1 g of cells in 25 mL of PBS or 1% (v/v) Triton X-100). RBCs in PBS and in Triton X-100 were used as a negative and positive control, respectively. The peptide solutions were prepared in PBS (0.01 M, pH 7.4) at concentrations ranging from 0.007 to 7 mM, and 20 4 of each peptide solution were added to 180 4 of 4% (w/v) RBCs in PBS. PAX-loaded nanofibers prepared at a ratio of 1:7.36 [PAX:TPGnRH consisting of 3 μmol (2.56 mg mL⁻¹) of PAX and 22.02 μmol (31.46 mg mL⁻¹) of TPGnRH] were diluted in PBS to the same range of concentrations as that for the TPGnRH. A range of PAX solutions were prepared in Cremophor EL and ethanol (1:1) at the same concentration of the PAX loaded within the nanofibers. The reasoning for the dilution of PAX in this mixture is because the available clinical formulation contains PAX dissolved in a 50% Cremophor EL and 50% dehydrated ethanol (1:1) to enhance drug. Then, the RBCs were incubated with the peptides or PAX for 1 hour at 37° C. After incubation, the cells were centrifuged at 1,200 g using a Haraeus Multifuge 3 S-R (Thermo Scientific, UK) during 10 minutes. The supernatant (100 μL) was transferred to a 96-well plate, and absorbance was measured at 590 nm. Percentage of haemolysis was calculated using Equation 1:

$$Haemolysis ( \% ) = \frac{{Abs}_{Sample} - {Abs}_{Negative Control}}{{Abs}_{Positive Control} - {Abs}_{Negative Control}} \times 100$$

Abs _{Negative Control} represents the absorbance of the RBCs in PBS and Abs _{Positive Control} the absorbance of the RBCs in 1% (v/v) Triton X-100. A control for Cremophor EL and ethanol mixture was also included.

Glu-GnRH shows no haemolytic toxicity at concentrations ranging 0.007-7 mM in rat RBCs. The TPGnRH and PAX-loaded TPGnRH cause haemolysis in a concentration-dependent manner with similar IC50 of 200 μM. Cremophor EL® and ethanol (1:1) formulation of PAX show haemolysis of 35.67% independently of the concentration of PAX, which is attributed to toxicity conferred by the solvents.

### Example 13

### Cytotoxicity of TPGnRH in Blood-Brain Barrier Cells

Human brain microvessel endothelial cells (hCMEC/D3) and cerebral astrocytes (SC-1800) were maintained in a humidified atmosphere of 5% CO₂, and the medium was refreshed every 2-3 days. hCMEC/D3 cells were cultured in endothelial basal medium-2 (EBM-2) supplemented with the EGM-2 Bullet Kit and 2% (v/v) human serum. SC-1800 were cultured in astrocytes basal medium (ABM) supplemented with AGM Bullet Kit and 3% (v/v) human serum. When cells reached ˜80% of confluency, cells were washed with HBSS and incubated with ˜2 mL of TrypLE Enzyme at 37° C. during 3 minutes. Subsequently, complete growth medium (4 mL) was added and cells were centrifuged using a C-28A centrifuge (Boeco, Hamburg, Germany) at 1,000 rpm for 5 minutes. Supernatant was discarded, and pellet was re-suspended in 1 mL of medium. The cell number was counted using Trypan Blue Exclusion Assay in the Countess II Automated Cell Counter (Thermo Scientific, Paisley, UK). hCMEC/D3 and SC-1800 cells were seeded at 20,000 cells/cm² in complete medium and allowed to attach overnight. Media was refreshed and cells were treated with TPGnRH peptide solutions in PBS (20 μL, 35-1400 μM TPGnRH) to achieve the final concentration of 3.5 to 140 μM (5 to 200 μg mL⁻¹) per well (20 μL of peptide into 180 μL of complete media). Cell metabolic activity was measured after 4 and 24 hours using (3-(4,5)-dimethylthiazol-2-yl)-2,5-diphenyltetrazolium bromide (MU) assay. At the specific time-point, the MU solution (20 μL at 5 mg mL⁻¹ solution in PBS) was added to each well and cells were incubated for 4 hours at 37° C. Subsequently, DMSO (100 μL) was added to dissolve the formazan crystals and absorbance was measured at 570 and 690 nm. Metabolic activity was calculated by subtracting the absorbance values at 690 nm to 570 nm to remove background, and dividing the values by the control to express as a percentage (%) of the control using the equation below. Untreated cells were used as a negative control.

$$Cell Metabolic Activity ( \% ) = \frac{( A b {s}_{570 nm Samp1e} - A b {s}_{690 nm Samp1e} ) \times 100}{( A b {s}_{570 nm {Control}^{-}} A b {s}_{690 nm Control} )}$$

TPGnRH nanofibers (3.5-140 μM) showed no cytotoxicity in hCMEC/D3 and SC-1800 cells at 4 and 24 hours of incubation.

### Example 14

### Permeability Across an In Vitro Blood-Brain Barrier Model

In order to test the permeability of TPGnRH nanofibers across the blood-brain barrier (BBB), a static contact co-culture was set up according to a previous method⁷. Briefly, human astrocytes (SC-1800, 25,000 cells/well) were seeded on the abluminal side of a 24-well plate Transwell polycarbonate membrane (3 μm of a pore size, Corning Star, Deeside, UK) coated with human fibronectin. Cells were allowed to attach for 3 days, and then human brain microvascular endothelial cells (hCMEC/D3, 75,000 cells/well) were seeded on the luminal side of the Transwell and co-cultured with astrocytes for five days with a daily change of the media. Transendothelial resistance was measured using EVOM voltohmmeter (World Precision Instruments, Berlin, Germany).

Permeability of diazepam, fluorescein (FITC)-dextran (3-5 KDa), PAX, PAX-loaded TPGnRH, and Texas Red (TR) labelled TPGnRH was evaluated from the luminal to the abluminal direction. Transwells were washed with HBSS and immersed into HBSS (600 μL in abluminal side). Diazepam (50 μg mL⁻¹), FITC-dextran (500 μg mL⁻¹), PAX (50 μg mL⁻¹), PAX-loaded TPGnRH (200 μg mL⁻¹ TPGnRH and 20 μg mL⁻¹ PAX) or TR-TPGnRH (200 μg mL⁻¹) solutions in HBSS (150 μL) were added to the luminal side of the Transwell and the co-cultures were incubated at 37° C. under stirring at 150 rpm (Heidolph Titramax 1000, Heidolph, Schwabach, Germany). Samples (100 μL) were collected from the abluminal side at specific time-points, and fresh HBSS (100 μL) was used to replace the volume from the abluminal compartments. Diazepam samples were analysed on Hypersil BDS C18 column (Phenomenex, 5 μm of pore size, 150×4.6 mm) using an isocratic method in an HPLC Agilent 1100 Series. Mobile phase consisted in acetonitrile, methanol and phosphate buffer (20 mM at pH 2.37) at a ratio of 27:10:63, and diazepam was eluted with a flow rate of 1.5 mL min-1 at 25° C. Injection volume was 30 μL, and detection was done at 230 nm. FITC-dextran was analysed in a plate reader spectrophotometer (BMG LABTECH, Ortenberg, Germany) λ_{exc} 485 and λₑₘ 520 in a black 96-well plate. PAX was analysed using reverse phase-HPLC with the gradient method described on Table 2. TR-TPGnRH was quantified in POLARstar Omega plate reader spectrophotometer with λ_{exc} 596 and λₑₘ 615 nm. The permeability coefficient (Pₐₚₚ) was determined using the equation:

$${P}_{a p p} = \frac{Δ {Q}_{r}}{{Δ}_{t}} \times \frac{{V}_{r}}{A \times {C}_{0} \times 60}$$

ΔQᵣ, is the flux of drug along time (μg mL⁻¹), Vᵣ is the volume of the abluminal side (mL), A is the area of the Transwell membrane, C₀ is the concentration of drug in the luminal side, and 60, the converser factor from minutes to seconds⁸.

TPGnRH was labelled with Texas Red (TR) according to the manufacturer's protocol. Endothelial cells were seeded (227,000 cells/cm²) in 24-well plates, allowed to attach for 24 hours, and then media was replaced by fresh media containing TR-TPGnRH at 50 μg mL⁻¹ in PBS. Cells were either incubated at 4 or 37° C. for 1 and 4 hours. At the end of a time-point, cells were washed with HBSS to remove the free nanofibers, incubated with 500 μL of TrypLE Express Enzyme and collected by centrifugation. The cell pellet was then re-suspended in PBS, and cells were analysed in BD FACS Calibur™ flow cytometer (BD Biosciences, Wiltshire, UK) acquiring at least 10,000 events. Data were analysed in FlowJo® Software 10.1. Unstained cells were used as a control in the flow cytometry settings.

Permeability studies using diazepam and FITC-dextran (3-5 kDa) confirmed the ability of the in vitro BBB model to distinguish mechanisms of permeation (transcellular and paracellular transport, respectively) (Table 8). Free PAX shows low permeation across the BBB model (Pₐₚₚ 0.19×10⁻⁶ cm s⁻¹), however when loaded within nanofibers, a Pₐₚₚ of 4.70×10⁻⁶ cm s⁻¹ was obtained. TR-TPGnRH presented a Papp of 6.0×10⁻⁶ cm s⁻¹.

TABLE 8
Permeability coefficients across the in vitro BBB model.

| | Pₐₚₚ (×10⁻⁶ cm s⁻¹) |
|---|---|
| Diazepam | 121.29 ± 24.81 |
| FITC-Dextran (3 to 5 kDa) | 3.28 ± 0.13 |
| Free Texas Red | 111.78 ± 24.30 |
| Texas Red-TPGnRH | 6.00 ± 0.07 |
| Free PAX | 0.19 ± 0.01 |
| PAX Loaded Texas Red-TPGnRH | 4.70 ± 0.18 |

Cell uptake studies indicated that TR-TPGnRH is uptaken by endothelial cells by an energy-dependent mechanism (0.18±0.05 versus 68.43±0.85% of the TR-TPGnRH⁺ cells at 4 and 37° C., respectively, at 4 hours of incubation).

### Example 15

### Receptor Binding Using Single Molecule Force Spectroscopy

The binding of the GnRH analogues to GnRH-R was assessed using single molecule force spectroscopy (SMFS) and Topography and Recognition (TREC) imaging based on AFM. AFM tips were functionalised with Glu-GnRH, goserelin acetate and TPGnRH as a monomer and nanofibers using a previously described method ^{9, 10}. Silicon nitride (6-20 pN/nm spring constant, Bruker, Mass., USA) and magnetically coated MACLevers tips (Keysight, Calif., USA) were used for SMFS and TREC studies, respectively. Briefly, both AFM tips were aminofunctionalised by incubating them with 3-aminopropyltriethoxysilane and triethylamine using vapour deposition, washed with chloroform, and incubated with a flexible heterobifunctional linker, NHS-PEG₁₈-acetal, in chloroform and triethylamine for 2 hours at room temperature. Subsequently, acetal groups were converted into aldehyde groups by immersing the tips in a 1% (w/v) citric acid solution for 10 minutes. Coupling of the peptide was achieved by immersing the tips into peptide solutions and 1 M of cyanoborohydride aqueous solution. Following an incubation of 2 hours, the free aldehyde groups were inactivated by ethanolamine for 10 minutes. AFM tips were washed with PBS three times and stored at 4° C. SMFS studies were conducted in living glioblastoma cells [U-87 MG, an immortalised cell line derived from a female patient (44-years old), UP-007, UP-029 and SEBTA-023, cell lines derived in house from biopsies] using Agilent 5500 AFM (Agilent Technologies, Inc., California, USA) in HBSS at room temperature. Hundreds (500 to 1000) of force-distance curves were collected for each set of measurements and each set of was performed with 2 to 3 different tips on 8-15 different cells, at random locations. Loading rates were calculated by multiplying the pulling velocity (v) with the effective spring constant (k_{eff}). Spring constants were determined by the thermal-noise method. The force-distance curves were acquired using PicoView 1.12 (Agilent Technologies, Inc., California, USA) and analysed in Matlab 8.1 software (MathWorks, Massachusetts, USA). TREC experiments were carried out in U-87 MG cells fixed with 0.25% (v/v) glutaraldehyde and all images were acquired in magnetic alternating current mode using the PicoTREC module on the Agilent 6000 ILM AFM (Agilent Technologies, Inc., California, USA) with tips with a nominal spring constant of 0.1 N/nm with a quality factor Q of ˜1 in liquid. Amplitude-distance curves were collected on a glass coverslip to adjust the free oscillation amplitude, and to determine the optimal amplitude reduction value for driving the feedback loop during the imaging. Free amplitude of cantilever oscillation was 30 nm and the excitation frequency was set to ˜9 kHz. TREC data were collected as 128×128 matrix with a line scan rate of 1 Hz, and feedback loop was coupled to the minima of the oscillations. The raw data was further analysed with the PicoScan 1.18 and Gwyddion software.

Blocking experiments were performed with different GnRH-R ligands (anti-GnRH-R antibody, Glu-GnRH, goserelin acetate, and TPGnRH). U-87 MG cells were analysed with tips functionalised with TPGnRH nanofibers and then the free ligands were added at a final concentration of 1 μg mL⁻¹ (anti-GnRH-R antibody) or 50 μg mL⁻¹ (Glu-GnRH, goserelin acetate and TPGnRH). U-87 MG cells treated with ligands were incubated for, at least, one hour at room temperature, and then the same cells were analysed with the same TPGnRH-functionalised tip at the same location in the cell.

The interaction of TPGnRH nanofibers with the GnRH-R expressed on living U-87 MG and UP-007 cells, was studied using SMFS. TPGnRH showed a binding probability of 13.2±0.9, 20.5±5.8, 9.5±2.5 and 17.04±3.1% in U-87 MG, UP-007, UP-029 and SEBTA-023, respectively. TPGnRH and Glu-GnRH illustrated similar binding force to the GnRH-R (30 and 50 pN) suggesting that both peptides bind to the same binding pocket within the GnRH-R. The lower dissociation rate is associated to a higher time of residence within the receptor and, possibly, a greater in vivo efficacy (Table 9). The blockage of the receptor with free GnRH-R ligands caused a significant reduction in the binding probability of TPGnRH attached to the AFM tip indicating the specificity of the peptide to the GnRH-Rs (Table 10). TREC imaging showed an irregular distribution of clusters of the GnRH-R (a diameter ˜10 to 90 nm, 48.0±24.84 nm) on the surface of U-87 MG cells. FIG. 3 shows TREC images of U-87 MG cells.

TABLE 9
Binding probability, binding force and dissociation rate of the
GnRH peptides to the GnRH-R expressed on glioblastoma cells
U-87-MG, UP-007, UP-029 and SEBTA-023. Mean ± SD.

| | Binding | Binding Force | Dissociation |
|---|---|---|---|
| | Probability (%) | (pN) | Rate (K_{off}) |
| U-87 MG | | | |
| Glu-GnRH | 13.3 ± 1.91 | 25.88 ± 0.94 | 1.29 ± 0.34 |
| Goserelin | 4.4 ± 0.94 | 29.88 ± 2.34 | 0.68 ± 0.08 |
| TPGnRH | 13.2 ± 1.64 | 30.00 ± 5.57 | 0.89 ± 0.10 |
| UP-007 | | | |
| Glu-GnRH | 12.84 ± 4.32 | 38.20 ± 9.16 | 1.52 ± 0.20 |
| Goserelin | 7.48 ± 0.76 | 34.00 ± 15.5 | 1.54 ± 0.12 |
| TPGnRH | 20.50 ± 5.80 | 50.80 ± 2.88 | 1.23 ± 0.13 |
| UP-029 | | | |
| Glu-GnRH | 6.93 ± 2.96 | 45.6 ± 4.64 | 1.66 ± 0.25 |
| TPGnRH | 9.51 ± 2.53 | 46.4 ± 9.45 | 2.03 ± 4.14 |
| SEBTA-023 | | | |
| Glu-GnRH | 4.93 ± 0.91 | 40.3 ± 11.70 | 1.22 ± 0.31 |
| TPGnRH | 17.04 ± 3.10 | 41.4 ± 3.85 | 2.49 ± 0.12 |

TABLE 10
Binding probability of TPGnRH nanofibers to U-87 MG cells,
before and after the addition of free GnRH-R ligands.

| | | Binding Probability (%) | | |
|---|---|---|---|---|
| Pre- | Anti-GnRH-R | | Goserelin | TPGnRH |
| Incubation | Antibody | Glu-GnRH | Acetate | Nanofibers |
| No | 11.3 | 12.6 | 16.2 | 14.7 |
| Yes | 5.6 | 3.4 | 3.8 | 4.5 |

### Example 16

### In Vitro Antitumour Assays

In vitro antitumour effects of TPGnRH and PAX-loaded TPGnRH were assessed in U-87 MG cells, UP-007, UP-029, SEBTA-023 MDA-MD-231 [triple negative (ER⁻, PR—, HER2⁻) breast cancer cell line] and SK-OV-3 (human ovarian carcinoma with a low expression of GnRH-R). Cells were seeded at 1,400 cells/cm² and allowed to attach and grow for 3 days. In experiments with TPGnRH nanofibers alone (to obtain the IC₅₀ value in each cell line), cells were treated with medium supplemented with the peptide solutions in PBS (0.7 to 700 μM) to achieve a final concentration of 0.07 to 70 μM per well. Media was replaced by fresh media every two days for 6 days. In assays with PAX-loaded TPGnRH, U-87 MG cells were treated with the TPGnRH nanofibers at 7 or 35 μM every two days for 4 days (2 doses). On day 4, PAX-loaded TPGnRH nanofibers containing 1 nM of PAX and 7 or 35 μM of TPGnRH were added and the effect on cell viability, proliferation, cell cycle, and apoptosis was assessed on day 6. Only for the MDA-MB-231 cells, on day 4, PAX-loaded TPGnRH nanofibers containing 10 nM of PAX and 7 or 35 μM of TPGnRH were added and the effect on cell viability, proliferation, cell cycle, and apoptosis was also assessed on day 6. The rationale for this experimental set up is that the pre-treatment with TPGnRH nanofibers reduces cell viability of glioblastoma cells, and one dose of the PAX-loaded TPGnRH nanofibers is enough to affect the remaining GnRH-R⁻ cells. In addition, the reduction in the doses of PAX results in the avoidance of excessive toxicity of PAX in the patients. TPGnRH nanofibers and PAX-loaded TPGnRH can be administered in cycles allowing time for the patients to recover from the PAX side-effect on white blood cells counts prior to another cycle of the PAX-loaded nanofibers. Cell metabolic activity was measured using MTT assay. At the end of each time-point, a MTT solution was added to each well at a final concentration of 0.5 mg mL⁻¹, and the cells were incubated for 4 hours. Afterwards, dimethyl sulfoxide (100 μL) was added to the wells to dissolve the formazan crystals and the absorbance was measured at 570 and 690 nm. The Bromodeoxyuridine (BrdU) ELISA Kit (ab126556, Abcam, Cambridge, UK) was used for the quantification of tumour cell proliferation. Cells were incubated with BrdU for 24 hours before the end of the pre-determined time-points. BrdU was quantified using the ELISA kit was used according to the manufacturer's instructions. A propidium iodide staining was used to identify the amount of cells in the interphases of cell cycle. At the pre-determined time-points, cells were washed twice with HBSS, incubated with TrypLE Enzyme, and collected by centrifugation. Cells were washed with ice-cold PBS and fixed in ice-cold 70% (v/v) ethanol and stored at −20° C. until the propidium iodide staining and flow cytometry analysis. Prior to flow cytometry analysis, the cells were washed with PBS, treated with RNase A (0.1 mg mL⁻¹) for 30 minutes at 37° C., and stained with propidium iodide (50 μg mL⁻¹) for 5 minutes. Cells were analysed in a BD FACS Calibur™ flow cytometer collecting at least 10,000 events. Apoptosis was measured using Annexin V-FITC Apoptosis Detection Kit (ab14085, Abcam). U-87 MG cells were washed with HBSS, incubated with the TrypLE Enzyme, and collected by centrifugation. Samples were stained according to the manufacturer's instructions and samples were analysed in a BD FACSCalibur™ flow cytometer acquiring at least 10,000 events. Unstained cells and cells only stained with the Annexin V-FITC or propidium iodide were used as controls. The intracellular cAMP was quantified using the Direct cAMP ELISA kit (ADI-900-066, Enzo, Exeter, UK). Cells were seeded at a density of 2,800 cells/cm² and after two days, cells were washed with serum-free medium, pre-treated with 3-isobutyl-1-methylxanthine (IBMX, 0.5 mM) for 15 minutes at 37° C. and treated with forskolin (FSK, 5 μM), either alone or in the presence of goserelin (1 μM), TPGnRH (7 or 35 μM) or PAX-loaded nanofibers (1 nM of PAX, 7 μM TPGnRH), for 15 minutes, at 37° C. At the end of the treatment, cells were lysed with hydrochloric acid (0.1 M) for 10 minutes and centrifuged for 10 minutes. cAMP was quantified using the ELISA kit according to the manufacturer's instructions. Cell uptake studies with U-87 MG cells were carried out as described above.

The IC₅₀ values of TPGnRH nanofibers were obtained in an immortalised glioblastoma cell line (U-87 MG) and cell lines derived from biopsies (UP-007, UP-029 and SEBTA-023). In addition, the IC50 was calculated for a cell line with a low expression of GnRH-R (SK-OV-3) (Table 11).

TABLE 11
IC₅₀ values of TPGnRH nanofibers in glioblastoma cell lines
(U-87-MG, UP-007, UP-029 and SEBTA-023) triple
negative breast cancer cells (MDA-MB-231) and cells with
a low expression of GnRH-R (SK-OV-3). Mean ± SD.

| Cells | Day 2 | Day 4 | Day 6 |
|---|---|---|---|
| U-87 MG | 28.48 ± 4.35 | 18.09 ± 3.07 | 10.34 ± 3.46 |
| UP-007 | 18.7 ± 4.83 | 11.51 ± 4.89 | 19.39 ± 4.74 |
| UP-029 | 14.27 ± 4.55 | 7.79 ± 4.47 | 2.2 ± 4.07 |
| SEBTA-023 | 13.06 ± 3.95 | 11.03 ± 3.82 | 5.6 ± 3.74 |
| MDA-MB-231 | 17.14 ± 1.03 | 40.46 ± 1.24 | 47.97 ± 1.33 |
| SK-OV-3 | 258.0 ± 58.67 | 170.5 ± 46.67 | 105.6 ± 23.94 |

The treatment of U-87 MG cells with the TPGnRH nanofibers (>7 μM, every two days, 6 days of treatment) was found to cause a significant reduction in cell proliferation with cell cycle arrest at the G2/M phase, and to trigger apoptosis. The TPGnRH nanofibers counteracted the forskolin-induced cAMP intracellular accumulation revealing that the effect of the nanofibers is mediated by the GnRH-R coupled to a G0 protein in glioma. When treated with TPGnRH (>7 μM, every two days, treatment for 4 days) and one dose of PAX-loaded nanofibers, the effects of TPGnRH nanofibers are combined with PAX causing a greater decrease in cell proliferation and an increase in the percentage of cells at G2/M (21.81±3.98 versus 65.59±2.99% for TPGnRH alone at 35 μM and PAX-loaded TPGnRH). FIG. 4 shows the effect of PAX-loaded nanofibers on U-87 MG and MDA-MB-231 cells.

### Example 17

### Cell Uptake Studies

The U-87 MG and SK-OV-3 cells were seeded at 20,000 cells/cm² in a 12-well plate (76,000 cells per well). Cells were allowed to attach overnight, and then the media was replaced by fresh media (900 μL) containing TR-TPGnRH (50 μg mL⁻¹ in PBS, 100 μL, pH 7.4). Subsequently, cells were either incubated at 4 or 37° C. for 1 and 4 hours. At the end of each time-point, cells were washed with HBSS twice to wash the free fibres, incubated with 500 μL TrypLE Enzyme at 37° C. for 3 minutes, and then collected by centrifugation at 1,000 rpm for 5 minutes using a C-28A centrifuge. Cells were then re-suspended in PBS (200 μL) and analysed in BD FACSCalibur™ flow cytometer acquiring at least 10,000 events. Unstained cells were used as a control in the flow cytometry settings.

Fluorescent microscopy and flow cytometry illustrated that TR-TPGnRH nanofibers are uptaken via U-87 MG cells by an energy-dependent mechanism (Table 12).

TABLE 12
Cell uptake of TR-TPGnRH nanofibers at 4 and 37° C. Mean ± SD.

| Time of Incubation | 4° C. | 37° C. |
|---|---|---|
| U-87 MG | | |
| 1 Hour | 20.51 ± 3.10 | 32.64 ± 5.04 |
| 4 Hours | 52.36 ± 10.20 | 91.12 ± 1.11 |
| SK-OV-3 | | |
| 1 Hour | 10.02 ± 0.52 | 13.35 ± 0.53 |
| 4 Hours | 52.47 ± 3.34 | 49.36 ± 4.66 |

### Example 18

### In Vivo Pharmacokinetic Studies

Peptide nanofibers (TPGnRH) were intravenously administered (5 mg mL⁻¹, 35 mg kg⁻¹) in sodium chloride (0.9% w/v, ˜150 μL) in BALB/c male mice (n=3). At 5 and 60 minutes, mice were killed and the blood and brain were harvested. Blood samples (0.5-0.7 mL per mouse) were collected into evacuated, sterile, spray coated with tripotassium ethylenediamine tetraacetic acid (3.6 mg), medical grade PET tubes. Plasma was separated from the blood by centrifugation (4,500 rpm for 15 min at 4° C., Hermle Z323 centrifuge, Hermle Labortechnik GmbH, Gosheim, Germany) and stored in −80° C. till analysed. Brain was weighted and homogenised with ice-cold PBS (1×, 7.4) using a 3 mL glass homogeniser. Plasma (100 μL) and brain homogenate (˜800 μL) were extracted with equal volumes of ice-cold acetonitrile three times and samples were dried using a centrifugal concentrator (SPD1010 SpeedVac System, ThermoSavant, Renfrewshire, United Kingdom) under vacuum over 2 hours and 4 hours respectively. Samples were orbitally agitated using a SciQuip Microplate Shaker attached to a tube adapter for 1.5-2 mL tubes (10 mm) for 10 minutes prior being centrifuged at 8,000 rpm for 5 minutes. Supernatants were pipetted in 0.2 mL amber vials and was quantified by reverse phase-HPLC. Analysis was conducted on Onyx Monolithic C18 column (4.6 mm×10+100 mm, 5 μm pore size) with a gradient method (Table 13) using an Agilent 1200 Series HPLC system (Agilent Technologies, Cheadle, UK). The PAX was eluted with a flow rate of 1.5 mL min⁻¹ at 30° C. Injection volume was 40 μL, and the detection was performed at 220 nm and 280 nm. The time of retention was 17.834 minutes for TPGnRH. Extraction efficiency was found to be 92.4±2.2% from plasma and 62.3±4.1% from brain tissue.

TABLE 13
Reverse phase-HPLC gradient method for TPGnRH
extracted from biological samples.

| | A: 0.1% (v/v) | B: 0.08% (v/v) |
|---|---|---|
| Time (minutes) | TFA in H₂O | TFA in ACN |
| 0 | 90 | 10 |
| 5 | 90 | 10 |
| 15 | 50 | 50 |
| 18 | 50 | 50 |
| 28 | 40 | 60 |
| 33 | 20 | 80 |
| 35 | 90 | 10 |

TABLE 14
TPGnRH plasma and brain levels after
intravenous administration (n = 3).

| TPGnRH | 5 minutes | 60 minutes |
|---|---|---|
| Plasma (μg/mL) | 544.8 ± 341.6 | 45.64 ± 10.12 |
| Brain (μg/g) | 3.08 ± 1.01 | 3.47 ± 0.08 |

Doses were well tolerated with no immediate signs of toxicity and gross alterations in liver, spleen, lungs and kidneys. Peptide nanofibers possessed a long circulation half-life (˜6% of dose in blood after 1 hour). Brain levels of the peptide increase from 5 to 60 minutes and the latter represent 0.47±0.01% of the injected dose (Table 14).

### Example 19

### Formulation of Peptide Nanofiber in 3D Printed Hydrogels

Peptide nanofibers were embedded in cellulose nanocrystals and sodium alginate hydrogels. Cellulose nanocrystals were synthesised by sulphuric acid degradation (45% w/v, 150 mls) of cellulose (15 g, Western Hemlock) at 50° C. over 90 minutes under stirring. Cold-deionised water was used to quench reaction and acid was removed by centrifugation (4,000 rpm, 10 minutes). Cellulose nanocrystals were dialysed (12-14 kDa for cytochrome C, Medicell Ltd) over 5 L with 6 changes over 24 hours. Resulting suspension was probe sonicated (400 watt, 23% amplitude, 10 minutes) in 50 mLs aliquots. The resulting suspension was centrifuged (4,000 rpm, 10 minutes) and supernatant was stored in the refrigerator. Cellulose nanocrystals (CNCs, 4.5% w/w, 2 g) were loaded onto a syringe attached to a three-way valve, where a syringe loaded sodium alginate (6% w/w in 4.6% glucose, 1.7 g). The CNCs were injected in the sodium alginate syringe and mixed 10 times (in each direction). Sample was collected in one syringe and a new syringe containing calcium chloride (0.1M, 0.9 mL). Sample was pushed into calcium chloride and mixed 5 times (in each direction) prior collecting the gel in one syringe and loading in cartridges. Peptide nanofibers (5 mg) were dispersed in 0.9 mL of 0.1M calcium chloride and mixed as above when needed. Release of paclitaxel loaded and TPGnRH gels in PBS (1×, 7.4, 6 mLs) was quantified as per HPLC method described previously (Example 7, Table 2). After an initial burst release, a controlled release profile is observed with gradual slow release over days. Resulting inks can be utilised for the production of nanofiber loaded implants for tumour patients.

TABLE 15
% release of TPGnRH nanofibers and paclitaxel loaded in
nanofibers 3D printed in cellulose-sodium alginate bioinks (n = 4).

| Time (Minutes) | % TPGnRH | % Paclitaxel |
|---|---|---|
| 0 | 0 | 0 |
| 1 | 15.4 ± 9.6 | 13.1 ± 15.6 |
| 5 | 15.4 ± 7.8 | 13.7 ± 5.6 |
| 10 | 16.3 ± 11.5 | 18.0 ± 10.6 |
| 15 | 15.4 ± 4.6 | 10.7 ± 2.3 |
| 30 | 16.4 ± 4.9 | 11.8 ± 5.2 |
| 45 | 14.7 ± 6.9 | 13.7 ± 8.4 |
| 60 | 19.7 ± 8.5 | 20.7 ± 9.6 |
| 90 | 20.6 ± 4.6 | 24.6 ± 5.1 |
| 120 | 21.8 ± 9.4 | 26.9 ± 9.4 |
| 240 | 20.6 ± 10.7 | 19.0 ± 10.9 |
| 360 | 22.6 ± 7.4 | 24.8 ± 7.3 |
| 1110 | 24.5 ± 7.9 | 28.0 ± 11.4 |
| 1440 | 28.5 ± 9.8 | 30.2 ± 9.8 |
| 2760 | 32.1 ± 9.7 | 37.1 ± 7.2 |

### REFERENCES

1 Bains, G., Patel, A. B. & Narayanaswami, V. (2011) Pyrene: a probe to study protein conformation and conformational changes. Molecules 16: 7909-7935.
2 Aguiar, J., Carpena, P., Molina-Bolivar, J. A. & Ruiz, C. C. (2003) On the determination of the critical micelle concentration by the pyrene 1:3 ratio method. Journal of Colloid and Interface Science 258: 116-122.
3 Sawada, T., Tsuchiya, M., Takahashi, T., Tsutsumi, H. & Mihara, H. (2012) Cell-adhesive hydrogels composed of peptide nanofibers responsive to biological ions. Polymer Journal 44: 651-657.
4 Meng, Q. et al. Tunable self-assembled peptide amphiphile nanosctructures. (2012) Langmuir 28: 5017-5022.
5 Biancalana, M. & Koide, S. (2010) Molecular mechanism of Thioflavin-T binding to amyloid fibrils. Biochimica et Biophysica Acta 1804: 1405-1412.
6 Liu, Y., Yang, Y., Wang, C. & Zhao, X. (2013) Stimuli-responsive self-assembling peptides made from antibacterial peptides. Nanoscale 5: 6413-6421.
7 Hatherell, K., Courard, P.-O., Romero, I. A., Weksler, B. & Pilkington, G. J. (2011) Development of a three-dimensional, all-human in vitro model of the blood-brain barrier using mono-, co-, and tri-cultivation Transwell models. Journal of Neuroscience Methods 199: 223-229.
8 Hakkarainen, J. J., Pajander, J., Laitinen, R., Suhonen, M. & Forsberg, M. M. (2012) Similar molecular descriptors determine the in vitro drug permeability in endothelial and epithelial cells. International Journal of Pharmaceutics 436, 426-443.
9 Zhang J., Wu G., Song C., Li Y., Qiao H., Zhu P., Hinterdorfer P., Zhang B., Tang J. (2012) Single molecular recognition force spectroscopy study of a luteinizing hormone-releasing hormone analogue as a carcinoma target drug. Journal of Physical Chemistry B 116(45): 13331-13337.
10 Zhang J., Chtcheglova L. A., Zhu R., Hinterdorfer P., Zhang B., Tang J. (2014) Nanoscale organization of human GnRH-R on human bladder cancer cells. Analytical Chemistry 86(5): 2458-2464.

1. A nanofiber comprising a peptide GPCR modulator conjugated to a lipophilic moiety wherein the peptide-lipophilic moiety conjugate comprises a poly(proline) type II helix structure.

2. The nanofiber according to claim 1, comprising one or more additional bioactive compounds, such as drugs or biomolecules, or imaging moieties or mixtures therefore.

3. The nanofiber according to claim 2, wherein the bioactive compound or imaging moiety is entrapped within the nanofiber, conjugated, or adsorbed onto the surface of the nanofiber.

4. The nanofiber according to claim 1, wherein the peptide is conjugated to the lipophilic moiety via a selectively cleavable link.

5. The nanofiber according to claim 1, wherein the GPCR modulator is selected from a gonadotrophin hormone releasing hormone (GnRH) receptor binding peptide, angiotensin 1-7, an opioid neuropeptide, neuropeptide S, a gastrin releasing peptide, orexin, dynorphin, detorphin I, oxytosin, vasopressin, leptin, enkephalin, met-enkephalin, tyr-enkephalin, urotensin II-Related Peptide (URP), urotensin II, vasoactive intestinal peptide, and secretin.

6. The nanofiber according to claim 1, wherein the peptide is less than 11 amino acids in length.

7. The nanofiber according to claim 1, wherein the peptide is a GnRH receptor binding peptide.

8. The nanofiber according to claim 7, wherein the peptide is selected from pyroGlu-His-Trp-Ser⁴-Tyr⁵-Gly⁶-Leu-Arg-Pro-Gly-NH₂ (GnRH), Glu-His-Trp-Ser⁴-Tyr⁵-Gly⁶-Leu-Arg-Pro-Gly-NH2 (Glu-GnRH) and Tyr-Gly-Leu-Arg-Pro-Gly-NH₂ (Tyr-GnRH).

9. The nanofiber according to claim 1, wherein the drug is selected from paclitaxel, docetaxel, temozolomide, doxorubicin, lomustine, etoposide, carmustine, buparvaquone, atovaquone and a polynucleotide, or mixtures thereof.

10. The nanofiber according to claim 1, comprising an imaging moiety selected from a visually infra-red ultra-violet detectable moiety, a spion, an MM contrast agent, a RAMAN tag and a deuterated moiety.

11. The nanofiber according to claim 1, wherein the lipophilic group comprises a saturated or unsaturated, branched or unbranched hydrocarbon group comprising at least 6 carbon atoms, more typically at least 8 or at least 16 carbon atoms.

12. The nanofiber according to claim 10, wherein the lipophilic group comprises a C₆-C₃₀ alkyl group, C₆-C₃₀ acyl group, a multicyclic hydrophobic group with more than one C₄-C₈ ring structure, a multicyclic hydrophobic group with more than one C₄-C₈ heteroatom ring structure, a polyaxa C₁-C₄ alkylene group, a hydrophobic polymer or lipidised D- or L amino acid modified at their N-terminal or side chain.

13. The nanofiber according to claim 1, wherein the lipophilic group is derived from a palmitoyl group, caprylic, capric, lauric, myristic, stearic, arachidic, cholic, deoxycholic or ursolic acids.

14. The nanofiber according to claim 1, wherein the linker is enzymatically cleavable or pH cleavable.

15. The nanofiber according to claim 1, comprising an overcoat of, or conjugated to one or more coating polymers.

16. The nanofiber according to claim 15, wherein the coating polymer is selected from sorbitan esters, polysorbates, polyethylene glycol, carbohydrates, glycol chitosan polymers, hyaluronic acid polymers and hyaluronic acid-chitosan or hyaluronic acid glycol chitosan copolymers, pullan, dextran, pectin, guar gum, alkyl glyceryl dextran, cellulose and cellulose derivatives or mixtures thereof.

17. A composition comprising a nanofiber according to claim 1, in combination with a gelling agent.

18. (canceled)

19. A pharmaceutical composition comprising a nanofiber according to claim 1, and further comprising a pharmaceutically acceptable carrier or excipient.

20. (canceled)

21. A method of treating a disease, comprising administering to a subject a pharmaceutically effective amount of a nanofiber according to claim 1.

22. A method according to claim 21, wherein the disease is a cancer, schizophrenia, obesity, pain, sleep disorder, psychiatric disease, neurodegenerative disease or infective disease.

23-25. (canceled)


SEQUENCE LISTING (3 sequences)

SEQ ID NO: 1 - protein, 10 aa, artificial
  gonadotrophin hormone releasing hormone receptor binding peptide
  misc_feature (1)..(1): Xaa = pyroGlu
XHWSYGLRPG

SEQ ID NO: 2 - protein, 10 aa, ARTIFICIAL
  Glu-GnRH
QHWSYGLRPG

SEQ ID NO: 3 - protein, 6 aa, artificial
  Tyr-GnRH
YGLRPG